(19) United States
(12) Patent Application Publication
(10) Pub. No.: US 2006/0099671 A1
(43) Pub. Date: May 11, 2006

(54) Methods and compositions for generating angiostatin

(75) Inventors: Gerald Soff, Skokie, IL (US); Stephen T. Gately, Palatine, IL (US); Przemyslaw Twardowski, Pasadena, CA (US)

Correspondence Address: JONES DAY, 222 EAST 41ST ST, NEW YORK, NY, 10017, US

(73) Assignee: Northwestern University

(21) Appl. No.: 11/318,939

(22) Filed: Dec. 22, 2005

Related U.S. Application Data
(63) Continuation of application No. 09/500,397, filed on Feb. 8, 2000.
Application No. 09/500,397 is a continuation-in-part of application No. 08/991,761, filed on Dec. 16, 1997, now Pat. No. 6,576,609.
Application No. 08/991,761 is a continuation-in-part of application No. 08/710,305, filed on Sep. 17, 1996, now Pat. No. 5,801,012.
Application No. 08/991,761 is a continuation of application No. PCT/US97/16539, filed on Sep. 17, 1997.

(51) Int. Cl. A61K 38/22; A61K 38/05; A61K 31/198; C12P 21/06

(52) U.S. Cl. 435/68.1; 514/12; 514/562; 514/18

(57) ABSTRACT

The invention provides methods of generating angiostatin in vitro comprising contacting plasminogen with a plasminogen activator and a sulfhydryl donor or contacting plasmin with a sulfhydryl donor. The invention also provides a method of treating angiogenic diseases by administering to an animal suffering from such a disease a sulfhydryl donor, a plasminogen activator, or a combination of a sulfhydryl donor and a plasminogen activator. The invention further comprises a composition for generating angiostatin comprising a sulfhydryl donor and a plasminogen activator. The invention also provides a container holding a sulfhydryl donor and/or a plasminogen activator, said container having a label thereon instructing administration of the sulfhydryl donor and/or plasminogen activator to an animal suffering from an angiogenic disease. The invention further provides plasminogen fragments whose N-terminal amino acid is the same as that of plasmin and whose C-terminal amino acid is located in kringle 5 and which inhibit angiogenesis, antibodies which bind selectively to these fragments, methods and kits for using the antibodies, methods and materials for making the fragments by recombinant DNA techniques, and a method of treating an angiogenic disease comprising administering an effective amount of one of the fragments. Finally, the invention provides a method of treating an angiogenic disease comprising administering a transgene coding for one of the fragments.

This application is a continuation-in-part of pending application Ser. No. 08/991,761, filed Dec. 15, 1997, which was a continuation-in-part of pending application Ser. No. 08/710,305, filed Sep. 17, 1996. Benefit of PCT application PCT/US97/16539 filed Sep. 17, 1997 is also claimed.

This invention was made, in part, with Government support received from the National Cancer Institute, grant number CA71875. The Government may have rights in the invention.

### FIELD OF THE INVENTION

This invention relates to angiostatin, an inhibitor of angiogenesis.

### BACKGROUND OF THE INVENTION

Angiostatin, a proteolytic fragment of plasminogen believed to consist of kringles 1 through 3 and all or part of kringle 4, is a potent inhibitor of angiogenesis and the growth of tumor cell metastases. O'Reilly et al., Cell, 79, 315-328 (1994); PCT application WO 95/29242. Angiostatin is found in vivo in tumor-bearing mice. O'Reilly et al., Cell, 79, 315-328 (1994); O'Reilly et al., Nature Med. 2, 689-692 (1996). The enzymatic mechanism by which angiostatin is generated in vivo remains unknown.

Angiostatin activity can be generated in vitro by limited elastase proteolysis of plasminogen. Sottrup-Jensen et al., in Progress in Chemical Fibrinolysis and Thrombolysis, 3, 191-209 (Davidson et al. eds. 1978). A recent abstract proposes that angiostatin is generated by macrophages infiltrating primary tumors and releasing elastase activity, which then cleaves plasminogen to form a protein having angiostatin activity. Dong et al., Proc. Am. Assoc. Cancer Res., 37 58 (1996). However, while limited elastase cleavage of plasminogen will yield a fragment or fragments having angiostatin activity, elastase will further digest the fragment(s) to inactive peptides, and therefore, is probably not the enzyme that generates angiostatin in vivo.

As noted above, angiostatin may be generated in vitro by limited elastase proteolysis of plasminogen. This method has several disadvantages. First, while elastase cleaves plasminogen to generate a fragment containing kringles 1-3, it is not known if this cleavage is at the normal sites where cleavage occurs to produce angiostatin in vivo. Therefore, the elastase-derived angiostatin may have altered in vivo processing with altered activity in humans. It may also be immunogenic if the sites of peptide cleavage are different from normal angiostatin.

A second means of producing angiostatin is by expressing the desired kringle domains of the plasminogen cDNA or gene in an expression vector in prokaryotic or eukaryotic cells. See PCT application WO 95/29242. This approach is also limited since the appropriate domains to express are not known. The product may also be immunogenic and may not be processed in humans as would be the product generated by cleavage of plasminogen by the normal in vivo enzymes.

Finally, angiostatin can be isolated from the body fluids of animals in which it is produced. See PCT application WO 95/29242. However, angiostatin cannot be produced in sufficient quantities for disease treatment in this manner, and the angiostatin may be contaminated with infectious agents when isolated from such sources.

Clearly a need exists for a method of producing native angiostatin in large quantities. “Native angiostatin” is defined herein to be the angiostatin produced in vivo or angiostatin, no matter how produced, which is the same as the angiostatin produced in vivo.

### SUMMARY OF THE INVENTION

The present invention provides such methods. These methods are based on the discovery that a conditioned culture medium (CCM) produced by culturing cancer cells, primary endothelial cells, smooth muscle cells or fibroblasts produces angiostatin when contacted with plasminogen or plasmin. The active factors in the CCM have been identified to be a plasminogen activator and a sulfhydryl donor. Thus, the angiostatin produced by the use of a plasminogen activator and sulfhydryl donor is the same as angiostatin produced in vivo, i.e., it is native angiostatin.

In one method of the invention for producing angiostatin in vitro, plasmin is contacted with a sulfhydryl donor to produce the angiostatin. The plasmin may be generated by contacting plasminogen with a plasminogen activator. Most conveniently, all of the reactants (plasminogen, plasminogen activator and sulfhydryl donor) can be contacted simultaneously to produce the angiostatin.

The angiostatin produced by this method, along with any remaining reactants, or angiostatin purified or partially purified from the reactants, may be administered to an animal, including a human, in need thereof. Animals in need of angiostatin are animals suffering from an angiogenic disease.

The invention further provides a composition for generating angiostatin. The composition comprises a sulfhydryl donor and a plasminogen activator. Two embodiments of the composition are CCM produced by culturing cells capable of producing plasminogen activator and a lysate of such cells.

The invention also provides a method of treating an angiogenic disease comprising administering to an animal suffering from such a disease an amount of a sulfhydryl donor effective to cause the conversion of plasmin to angiostatin. The plasmin may be that produced by endogenous plasminogen activator(s) from endogenous plasminogen. Alternatively, the method may further comprise administering an effective amount of plasmin. In yet other embodiments, a plasminogen activator may be administered to the animal to produce the plasmin from endogenous plasminogen or from an effective amount of administered plasminogen.

The invention also provides a method of treating an angiogenic disease comprising administering to an animal suffering from such a disease an amount of a plasminogen activator effective to cause the conversion of plasminogen (endogenous plasminogen or administered plasminogen) to plasmin. Endogenous or administered sulfhydryl donors then cause the conversion of the plasmin to angiostatin.

The invention further provides a container holding a plasminogen activator, alone or in combination with sulfhydryl donor. The container has a label thereon instructing administration of the plasminogen activator or the combination of the plasminogen activator and sulfhydryl donor to an animal suffering from an angiogenic disease. The invention also provides a container holding a sulfhydryl donor with a label thereon instructing administration of the sulfhydryl donor in an amount effective to cause the conversion of plasmin to angiostatin.

The invention also provides a protein having the following characteristics: (a) it is a fragment of plasminogen; (b) its N-terminal amino acid is the same as the N-terminal amino acid of plasmin; (c) its C-terminal amino acid is in kringle 5; and (d) it inhibits angiogenesis. In one embodiment, the protein is native angiostatin. The invention further provides a DNA molecule coding for the protein, the DNA molecule operatively linked to expression control sequences, a host cell comprising the DNA molecule operatively linked to expression control sequences, and a method of producing the protein comprising culturing the host cell.

The protein may be used to treat angiogenic diseases by administering an effective amount of the protein to an animal suffering from such a disease. An animal suffering from such a disease may also be treated by administering to it a transgene coding for the protein. Preferably, the protein coded for by the transgene is native angiostatin.

Finally, the invention provides an antibody which binds selectively to the protein. Such an antibody may be used to purify the protein from materials containing it. Also, such an antibody which binds selectively to native angiostatin may be used in methods and kits to detect or quantitate native angiostatin.

### BRIEF DESCRIPTION OF THE DRAWINGS

FIG. 1A: Western blot showing conversion of plasminogen and plasmin to angiostatin by serum-free conditioned medium (SFCM) produced by PC-3 cells. Lane 1, molecular weight standard; lane 2, human plasminogen; lane 3, human plasminogen incubated overnight at 37° C. in non-conditioned RPMI; lane 4, human plasminogen incubated overnight at 37° C. in SFCM from PC-3 cells; lane 5, human plasmin incubated in non-conditioned RPMI; lane 6, human plasmin incubated in SFCM produced by PC-3 cells.

FIG. 1B: Western blot showing that the generation of angiostatin from plasminogen was time dependent. PC-3 SFCM was incubated with plasminogen and, at the time-points indicated, aliquots were removed and snap frozen prior to western blot analysis. Trace generation of angiostatin was first observed at 3 hours, and complete conversion was noted at 24 hours.

FIG. 1C: Western blots showing that the generation of angiostatin by PC-3 SFCM was concentration dependent. SFCM was diluted with various amounts of fresh RPMI as indicated and incubated with plasminogen for 24 hours.

FIG. 1D: Graph illustrating the relationship of angiostatin generation to the amount of SFCM. The relative angiostatin signal was quantitated by scanning densitometer with background subtraction. At 18 hours incubation, there was a linear relationship between the amount of angiostatin generated and the amount of PC-3 SFCM present in the reaction mixture.

FIG. 2: Western blots after affinity purification of angiostatin generated by incubation of plasminogen with SCFM produced by PC-3 cells. Lane 1, molecular weight standards; lane 2, human plasminogen incubated overnight at 37° C. in non-conditioned RPMI; lane 3, angiostatin produced by incubation of plasminogen with PC-3 SCFM and then affinity purified on lysine-sepharose and detected on western blot by staining with Coomassie blue; lane 4, angiostatin produced by incubation of plasminogen with PC-3 SCFM and then affinity purified on lysine-sepharose and detected on western blot using the monoclonal antibody K1-3 to kringles 1-3.

FIGS. 3A-B: Graphs showing that angiostatin produced by incubating plasminogen with PC-3 SCFM inhibits in vitro steps critical for angiogenesis. FIG. 3A: Endothelial cell proliferation. The data are mean±standard deviation. FIG. 3B: Basic fibroblast growth factor (bFGF)-induced migration. Background migration without the inducer and in the presence of stimulatory bFGF are indicated. Toxicity was measured in parallel by trypan blue exclusion and was <10% at all concentrations.

FIGS. 4A-B: Photographs showing that angiostatin produced by incubating plasminogen with PC-3 SCFM inhibits human endothelial cell tube formation in vitro. Human umbilical vein endothelial cells (HUVEC) were plated on gels of Matrigel in 24-well dishes and then were treated with 15 μg/ml of angiostatin produced using PC-3 SFCM in non-conditioned RPMI. FIG. 4A: Control HUVEC form branching, interconnecting networks. FIG. 4B: By contrast angiostatin produced using PC-3 SFCM caused a significant disruption of the tube network.

FIGS. 5A-B: Photographs showing the inhibition of angiogenesis in vivo by angiostatin produced using PC-3 SCFM. FIG. 5A: A hydron pellet (indicated by the arrow) containing bFGF induced a positive neovascular response 7 days after implantation. FIG. 5B: By contrast, no vessels are observed approaching a hydron pellet containing bFGF and 10 μg/ml angiostatin produced using PC-3 SFCM (indicated by the arrow).

FIG. 6: Western blot showing that the batch eluate from Reactive Red 120-Agarose generates angiostatin when combined with Reactive Red 120-Agarose flow-through, RPMI or RPMI amino acids. Lane 1—SFCM+plasminogen; Lane 2—Reactive Red 120-Agarose flow-through +plasminogen; Lane 3—Reactive Red 120-Agarose batch eluate after dialysis to TBS+plasminogen; Lane 4—dialyzed batch eluate+Reactive Red 120-Agarose flow-through +plasminogen; Lane 5—dialyzed batch eluate+RPMI+plasminogen; Lane 6—dialyzed batch eluate+RPMI vitamin mix+plasminogen; Lane 7—dialyzed batch eluate+RPMI amino acid mix+plasminogen; Lane 8—dialyzed batch eluate+RPMI vitamin mix and amino acid mix+plasminogen; Lane 9—plasminogen+unconditioned RPMI.

FIG. 7: Graph showing that urokinase-type plasminogen activator (u-PA) activity and plasminogen-angiostatin converting activity (PACA) co-elute on a gradient elution from Hi-Q anion exchange column. Optical density readings at 280 nm demonstrated several protein peaks. u-PA activity was determined by measuring the cleavage of a chromogenic peptide substrate for plasmin (Val-Leu-Lys p-NA) at 405 nm. The peak fractions were assayed for PACA by western blot.

FIG. 8: Western blot showing that addition of u-PA and plasminogen to boiled Reactive Red 120-Agarose flow-through or fresh RPMI medium generated angiostatin. Lane 1—Reactive Red 120-Agarose flow-through+plasminogen; Lane 2—Reactive Red 120-Agarose flow-through+plasminogen+u-PA; Lane 3—Reactive Red 120-Agarose boiled flow-through+plasminogen; Lane 4—Reactive Red 120-Agarose boiled flow-through+plasminogen+u-PA; Lane 5—unconditioned RPMI+plasminogen; Lane 6—unconditioned RPMI+plasminogen+u-PA.

FIG. 9: Western blot showing that the Reactive Red 120-Agarose flow-through produces angiostatin in the presence of plasminogen activators. Lane 1—Reactive Red 120-Agarose flow-through+plasminogen; Lane 2—Reactive Red 120-Agarose flow-through+plasminogen+u-PA; Lane 3—Reactive Red 120-Agarose flow-through+plasminogen+t-PA.

FIG. 10: Western blot showing the production of angiostatin by u-PA and glutathione. Lane 1—plasminogen+u-PA; Lane 2—plasminogen+u-PA+5 μM glutathione; Lane 3—plasminogen+u-PA+50 μM glutathione; Lane 4—plasminogen+u-pA+100 μM glutathione; Lane 5—plasminogen+u-PA+boiled 5 μM glutathione; Lane 6—plasminogen+u-PA+boiled 50 μM glutathione; Lane 7—plasminogen+u-PA+boiled 100 μM glutathione.

FIG. 11: Western blot showing that the combination of u-PA and D-penicillamine produces angiostatin. Lane 1—plasminogen+100 μM D-penicillamine; Lane 2—plasminogen+u-PA+100 μM D-penicillamine; Lane 3—plasminogen+u-PA+1.0 mM D-penicillamine.

FIG. 12: Western blot showing the production of angiostatin by u-PA, t-PA and streptokinase. The abbreviations used in the figure have the following meanings:

PLG=human plasminogen;

uPA=urokinase-type plasminogen activator;

tPA=tissue-type plasminogen activator;

SK=streptokinase;

+=with N-acetyl-L-cysteine; and

−=without N-acetyl-L-cysteine.

FIG. 13: Western blot showing the production of plasmin from plasminogen and the production of angiostatin from the pre-formed, purified plasmin. Lane 1—plasminogen+u-PA-Sepharose; Lane 2—purified plasmin+100 μM N-acetyl-L-cysteine.

FIG. 14: Graph of mean primary tumor size (mm³) for days 0-21 for control mice and mice treated with N-acetyl-L-cysteine (NAC) or NAC+urokinase-type plasminogen inhibitor (uPA).

FIG. 15: Western blot showing the production of angiostatin by N-acetyl-L-cysteine (NAC) in vivo. Lane 1—plasma (diluted 1:20) from control mouse #2; Lane 2—plasma (diluted 1:20) from control mouse #3; Lane 3—plasma (diluted 1:20) from first mouse receiving affinity-purified, cell-free angiostatin; Lane 4—plasma (diluted 1:20) from second mouse receiving affinity-purified, cell-free angiostatin; Lane 5—plasma (diluted 1:20) from NAC-treated mouse #1; Lane 6—plasma (diluted 1:20) from NAC-treated mouse #2; Lane 7—plasma (diluted 1:20) from NAC-treated mouse #3; and Lane 8—affinity-purified, cell-free angiostatin.

FIG. 16: A diagram of human plasminogen showing the amino acid sequence of the complete molecule after cleavage of the signal peptide (not shown) (taken from Molecular Basis of Thrombosis and Hemostasis (High and Roberts, eds. 1995)). Kringles 1-5 (K1-K5) are indicated. The cleavage sites between residues 77 and 78 and residues 561 and 562 needed for activation of plasminogen to plasmin are indicated by filled arrows. The unfilled arrows represent the positions of introns in the gene. The locations of the N-linked oligosaccharide at position 289 and the O-linked glycan at position 346 are also indicated.

The * indicate members of the catalytic triad of plasmin (His603, Asp646 and Ser741).

FIG. 17A. A Western blot of plasma samples from a patient with a mesothelioma of the right hemi-thorax (Case #2—see Table 4 below) treated with urokinase alone (Cycle 2) and subsequently with captopril and urokinase (Cycle 5). Lane 1: Cycle 2, Day 1, Pre-Treatment. Lane 2: Cycle 2, Day 3, Post-Treatment. Lane 3: Cycle 5, Day 1, Pre-Treatment. Lane 4: Cycle 5, Day 3, Post-Treatment. A “Cycle” for this patient refers to the three days of treatment plus the days off treatment until the therapy was begun again (see Table 4 below).

FIG. 17B. Graph of cell numbers versus various amounts of the lysine-binding fractions of platelet-poor plasma samples taken from Case #2 in Cycle 2, Day 3 (solid bars) and Cycle 5, Day 3 (stippled bars) in a cell proliferation assay.

### DETAILED DESCRIPTION OF THE PRESENTLY PREFERRED EMBODIMENTS

The invention provides in vitro methods of generating native angiostatin. One such method comprises contacting plasminogen with a plasminogen activator and a sulfhydryl donor. All three of the reactants may be combined simultaneously. Alternatively, the plasminogen may be contacted with a plasminogen activator to produce plasmin, and the plasmin then contacted with a sulfhydryl donor to produce the angiostatin. The plasmin may be at least partially purified prior to contacting it with the sulfhydryl donor. Indeed, angiostatin can be produced directly from plasmin, however made, by contacting the plasmin with a sulfhydryl donor.

The plasminogen may be from any animal species. Preferably, however, plasminogen from the species of animal to be treated with the angiostatin is used to avoid immune reactions upon administration of the angiostatin. Thus, if a human is to be treated with the angiostatin, human plasminogen is preferably used.

Methods of making plasminogen are well known in the art. Plasminogen may also be purchased commercially. Preferably the plasminogen is prepared by recombinant DNA or other techniques that avoid the inclusion of infectious agents in the plasminogen preparation.

All types of plasminogen activators may be used, including urokinase-type plasminogen activators, tissue-type plasminogen activators and streptokinase. The plasminogen activator may be from any animal species. Methods of making plasminogen activators are well known in the art, and many plasminogen activators are available commercially. Preferably the plasminogen activator is prepared by recombinant DNA or other techniques that avoid the inclusion of infectious agents in the plasminogen activator preparation.

The plasminogen is contacted with the plasminogen activator in amounts and under conditions effective to cause the conversion of the plasminogen to plasmin. These amounts and conditions are known or can be determined empirically as is known in the art. In particular, from about 1 ng/ml to about 1 μg/ml of urokinase plasminogen activator for each microgram of plasminogen in a 1 ml reaction have been found to give complete conversion of plasminogen to plasmin after about 24 hours of incubation at 37° C.

Any sulfhydryl donor may be used. Sulfhydryl donors are well known and are available commercially. Suitable sulfhydryl donors include L-cysteine, D-cysteine, DL-cysteine, N-acetyl-L-cysteine, reduced glutathione, D-penicillamine and captopril. The sulfhydryl donor is believed to reduce or alter disulfide bond formation in the plasminogen, and/or the plasmin, and/or the angiostatin, and/or an intermediate product.

The sulfhydryl donor is contacted with the plasmin, alone or in the presence of the plasminogen and plasminogen activator, in amounts and under conditions effective to cause the conversion of the plasmin to angiostatin. These amounts and conditions can be determined empirically as is known in the art. In particular, from about 10 μM to about 1 mM of sulfhydryl donor for each microgram of plasmin in a 1 ml reaction have been found to give complete conversion of plasmin to angiostatin after about 24 hours of incubation at 37° C.

Plasmin may be generated from plasminogen by a plasminogen activator as described above. The plasmin may be purified from the reactants prior to contacting it with the sulfhydryl donor. Methods of purifying plasmin are known in the art (see, e.g., Example 4). Plasmin purchased commercially or prepared in other ways may also be used to produce angiostatin by contacting the plasmin with a sulfhydryl donor as described above.

The invention further provides a composition for generating angiostatin. The composition comprises a plasminogen activator and a sulfhydryl donor as described above. The plasminogen activator and sulfhydryl donor may be contained in any physiologically-acceptable solution (e.g., saline, buffers, culture medium) or may be present in crystalline or lyophilized form. Compositions suitable for therapeutic use are described below.

The composition may be a conditioned culture medium (CCM) prepared by culturing cells capable of producing plasminogen activator. Malignant animal cells, human and non-human, which express a plasminogen activator can produce CCM capable of converting plasminogen and plasmin into angiostatin. Suitable malignant cells include human prostate carcinoma cell lines PC-3, DU-145, LN-CaP, human breast carcinoma cell lines MDA-MB-231 and MCF-7, human glioma cell lines U-373, U-118, A-172, and U-87, and mouse melanoma cell line B16F10. Many non-malignant animal cells are known to produce is plasminogen activator. Suitable non-malignant cells include primary endothelial cells (e.g., bovine aortic endothelial cells), smooth muscle cells (e.g., bovine smooth muscle cells), and fibroblasts. In addition, bacterial cells are known which produce plasminogen activator (e.g., streptokinase), and cells of any type can be transformed by recombinant DNA techniques to produce plasminogen activator. Suitable cells and cell lines are well known in the art and may be obtained commercially, from cell depositories, and bymethods well known in the art.

Suitable culture conditions for these cells are also well known in the art. The culture medium used must contain a sulfhydryl donor, or a sulfhydryl donor may be added to the CCM after it is produced. Suitable culture media include those available commercially, such as RPMI, DMEM, etc. The CCM may be produced by simply culturing the cells under normal culture conditions for a sufficient time to produce CCM capable of converting plasminogen or plasmin to angiostatin. This time can be determined empirically. In particular, it has been found that culturing the mammalian cells for 24-72 hours after a monolayer has formed at 37° C. is sufficient.

Alternatively, or in addition, the cells can be lysed after culturing for a time sufficient to allow synthesis of plasminogen activator. This time can be determined empirically, but culturing the cells until a monolayer has formed should be sufficient. The lysate can be used to convert plasminogen and plasmin to angiostatin.

The angiostatin produced by these methods may be purified from the reaction mixture. Methods of protein purification are well known in the art. In particular, angiostatin may be purified by affinity chromatography using lysine-Sepharose. Residual plasmin activity should be removed with, e.g., soybean trypsin inhibitor-Sepharose, aprotinin-Sepharose, or other affinity chromatography procedures that remove serine proteases or the plasmin catalytic domain. The angiostatin may also be purified from the reaction mixture using an antibody that binds selectively to it (see below).

The angiostatin produced by these methods (native angiostatin) has been characterized. It reacts with a monoclonal antibody specific for kringles 1-3 of plasminogen and has been found to inhibit angiogenesis as assessed by a variety of tests in vitro and in vivo.

It has also been found to have the N-terminal sequence of plasmin. For angiostatin produced from human plasminogen, the N-terminal sequence has been found to be Lys Val Tyr Leu Ser Glu Cys Lys Thr Gly [SEQ ID NO:1]. The sequences of the plasmin of other animals are known. Thus, native angiostatin of a particular animal would have the same N-terminal sequence as the plasmin of that animal.

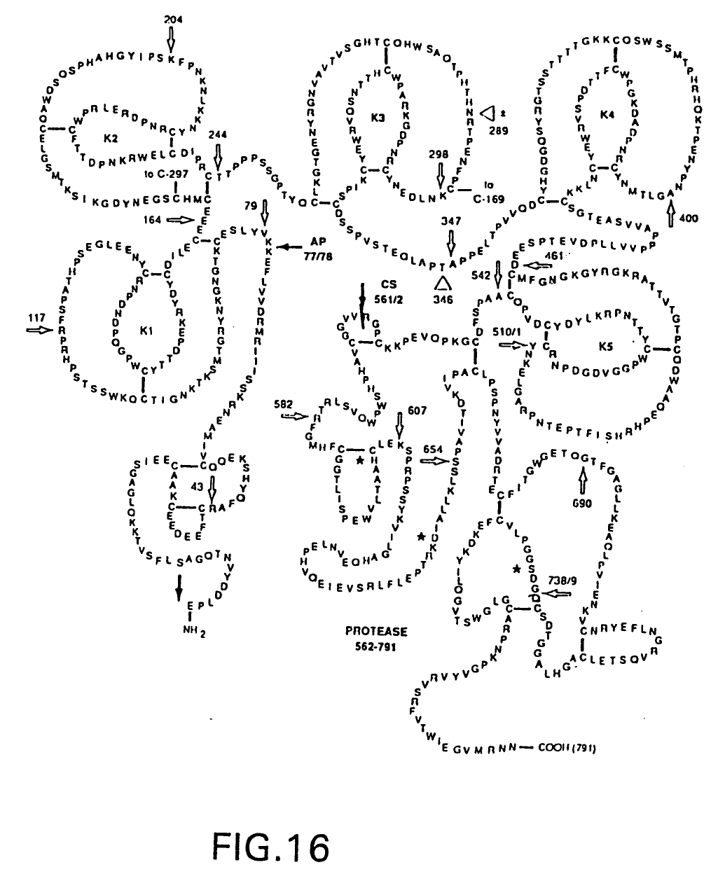

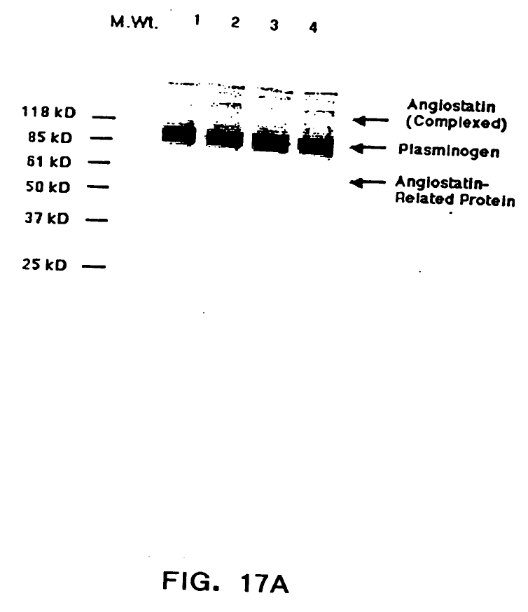

Quite surprisingly, native angiostatin has been found to have its C-terminal amino acid located in kringle 5. In particular, angiostatin produced from human plasminogen has been found to have the C-terminal sequence Cys Tyr Thr Thr Asn Pro Arg [SEQ ID NO:4] or Cys Tyr Thr Thr Asn Pro Arg Lys [SEQ ID NO:5] (see Example 6). These C-terminal sequences would result from a cleavage after amino acid 529 or 530 of plasminogen (see FIG. 16), which are known plasmin cleavage sites. Thus, native human angiostatin comprises most of kringle 5 (see FIG. 16), which is consistent with its molecular weight of 50-60 kD on polyacrylamide gel electrophoresis under non-reducing conditions.

These findings were surprising because it had been thought that angiostatin contained kringles 1-3 and part or all of kringle 4 (see Background section). Also, it has been shown that a molecule consisting of kringles 1-4, while active, was less active than a molecule consisting of kringles 1-3 (see PCT application WO 96/35774). Thus, it was unanticipated that native angiostatin would include any portion of kringle 5. It was particularly unanticipated that native angiostatin would include most of kringle 5.

It is now expected that plasminogen fragments, other than native angiostatin, including at least a portion of kringle 5 will possess angiostatin activity (i.e., will inhibit angiogenesis). Preferably the plasminogen fragment comprises the majority of kringle 5. More preferably the plasminogen fragment comprises most of kringle 5. As used herein “majority of kringle 5” means at least 50% of kringle 5 (e.g., at least 40 amino acids for human kringle 5), and “most of kringle 5” means at least 75% of kringle 5 (e.g., at least 60 amino acids for human kringle 5). Of course, the plasminogen fragment is most preferably native angiostatin for the reasons given above.

The sequences of plasminogens from other animals are known (available from, e.g., GenBank). The sequences of human [SEQ ID NO:6], bovine [SEQ ID NO:7], canine [SEQ ID NO:8], western European hedgehog [SEQ ID NO:9], horse [SEQ ID NO:10], rhesus monkey [SEQ ID NO:11], mouse [SEQ ID NO:12], and pig [SEQ ID NO:13] plasminogen are given below in the Sequence Listing (downloaded from SWISS-PROT Protein Sequence Database). Native angiostatin for a particular animal would include most of kringle 5 of that animal's plasminogen and would have a C-terminal sequence corresponding to the C-terminal sequences of human native angiostatin given above. Indeed, a review of these sequences showed that the sequence immediately after the cleavage sites in human plasminogen that produce native angiostatin (SEQ ID NO:2 and SEQ ID NO:3; see Example 6 below) is conserved in all of these plasminogen sequences (see the amino acids highlighted in bold in the Sequence Listing).

As can be observed from the Sequence Listing, the sequences of canine [SEQ ID NO:8] and horse [SEQ ID NO:10] plasminogens contain only a single kringle domain. This single kringle domain is considered a kringle 5 domain by homology to other kringle 5 domains, and it contains the conserved sequence (see highlighted amino acids in the Sequence Listing) found in the kringle 5 domains of the other plasminogens. Thus, the invention includes plasminogen fragments of canine and horse plasminogens and of any other plasminogen containing a kringle 5 domain.

Plasminogen fragments of the invention (those having the N-terminal sequence of plasmin and having their C-terminal amino acids located in kringle 5) can be produced by recombinant DNA methods. Preferably the plasminogen fragment is native angiostatin. Most preferably the plasminogen fragment is native human angiostatin. Recombinant DNA methods and suitable host cells, vectors and other reagents for use therein, are well known in the art.

The selection of a particular host cell for production of a plasminogen fragment of the invention is dependent upon a number of factors recognized by the art. These include, for example, compatibility with the chosen expression vector, toxicity of the plasminogen fragment to the cell, rate of transformation, expression characteristics, bio-safety, and costs. A balance of these factors must be struck with the understanding that not all hosts may be equally effective for the expression of a particular plasminogen fragment.

Eukaryotic host cells are preferred for making the plasminogen fragments of the invention. Within the above guidelines, useful eukaryotic host cells include yeast and other fungi, animal cell lines, animal cells in an intact animal, insect cells, and other eukaryotic host cells known in the art.

The host cells may be transformed with a vector comprising DNA encoding a plasminogen fragment of the invention. On the vector, the coding sequence must be operatively linked to expression control sequences.

As used herein “operatively linked” refers to the linking of DNA sequences in such a manner that the plasminogen fragment will be expressed. Preferably the linking, including the order of the sequences, the orientation of the sequences, and the relative spacing of the various sequences, is performed so that optimum expression is obtained.

The expression control sequences must include a promoter. The promoter used in the vector may be any sequence which shows transcriptional activity in the host cell and may be derived from genes encoding homologous or heterologous proteins and either extracellular or intracellular proteins. However, the promoter need not be identical to any naturally-occurring promoter. It may be composed of portions of various promoters or may be partially or totally synthetic. Guidance for the design of promoters is provided by studies of promoter structure such as that of Harley and Reynolds, Nucleic Acids Res., 15, 2343-61 (1987). Also, the location of the promoter relative to the transcription start may be optimized. See Roberts, et al., Proc. Natl. Acad. Sci. USA, 76, 760-4 (1979). The promoter may be inducible or constitutive, and is preferably a strong promoter. By “strong,” it is meant that the promoter provides for a high rate of transcription in the host cell.

In the vector, the coding sequences must be operatively linked to transcription termination sequences, as well as to the promoter. The coding sequence may also be operatively linked to expression control sequences other than the promoters and transcription termination sequences. These additional expression control sequences include activators, enhancers, operators, stop signals, cap signals, polyadenylation signals, 5′ untranslated sequences, and other sequences and signals involved with the control of transcription or translation.

The consensus sequence for the translation start sequence of eukaryotes has been defined by Kozak (Cell, 44,283-292 (1986)) to be: C(A/G)CCAUGG. Deviations from this sequence, particularly at the −3 position (A or G), have a large effect on translation of a particular mRNA. Virtually all highly expressed mammalian genes use this sequence. Highly expressed yeast mRNAs, on the other hand, differ from this sequence and instead use the sequence (A/Y)A(A/U)AAUGUCU (Cigan and Donahue, Gene, 59, 1-18(1987)). These sequences may be altered empirically to determine the optimal sequence for use in a particular host cell.

DNA coding for a plasminogen fragment of the invention may prepared using standard methods such as those described in Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). In particular, clones coding for plasminogen are known. See, e.g., GenBank PCT application WO 95/29242; Browne et al., Fibrinolysis, 5, 257-260 (1991). Other clones may be identified by methods known in the art. The clones, whether known or newly-identified, may be modified to code for a plasminogen fragment of the invention by methods known in the art.

The coding sequences may, alternatively, be synthesized using standard techniques that are well known in the art using the known plasminogen sequences. For instance, DNA sequences may be synthesized by phosphoamidite chemistry in an automated DNA synthesizer, purified, annealed, ligated and cloned into suitable vectors. Chemical synthesis is preferable for several reasons.

First, chemical synthesis is desirable because codons preferred by the host in which the DNA sequence will be expressed may be used to optimize expression. Not all of the codons need to be altered to obtain improved expression, but greater than 50%, most preferably at least about 80%, of the codons should be changed to host-preferred codons. The codon preferences of many host cells are known. See Maximizing Gene Expression, pages 225-85 (Reznikoff & Gold, eds., 1986). The codon preferences of other host cells can be deduced by methods known in the art.

The use of chemically synthesized DNA also allows for the selection of codons with a view to providing unique or nearly unique restriction sites at convenient points in the sequence. The use of these sites provides a convenient means of constructing the synthetic coding sequences. In addition, if secondary structures formed by the messenger RNA transcript or other destabilizing sequences interfere with transcription or translation, they may be eliminated by altering the codon selections.

Chemical synthesis also allows for the use of optimized expression control sequences with the DNA sequence coding for a plasminogen fragment. In this manner, optimal expression of the plasminogen fragments can be obtained. For instance, as noted above, promoters can be chemically synthesized and their location relative to the transcription start optimized.

DNA coding for a signal or signal-leader sequence may be located upstream of the DNA sequence encoding the plasminogen fragment. A signal or signal-leader sequence is an amino acid sequence at the amino terminus of a protein which allows the protein to which it is attached to be secreted from the cell in which it is produced. Suitable signal and signal-leader sequences are well known. Although secreted proteins are often easier to purify, expression levels are generally lower than those that can be obtained in the absence of secretion.

Vectors for expressing the plasminogen fragments may be any vector which may conveniently be subjected to recombinant DNA procedures and which is capable of expressing a plasminogen fragment in the selected host cell. The vector used to transform the host cells may have one or more replication systems which allow it to replicate in the host cells. In particular, when the host is a yeast, the vector should contain the yeast 2u replication genes REP 1-3 and origin of replication.

Alternatively, an integrating vector may be used which allows the integration into the host cell's chromosome of the sequence coding for a plasminogen fragment of the invention. Although the copy number of the coding sequence in the host cells would be lower than when self-replicating vectors are used, transformants having sequences integrated into their chromosomes are generally quite stable.

When the vector is a self-replicating vector, it is preferably a high copy number plasmid so that high levels of expression are obtained. As used herein, a “high copy number plasmid” is one which is present at about 100 copies or more per cell. Many suitable high copy number plasmids are known.

The vector desirably also has unique restriction sites for the insertion of DNA sequences and a sequence coding for a selectable or identifiable phenotypic trait which is manifested when the vector is present in the host cell (“a selection marker”). If a vector does not have unique restriction sites, it may be modified to introduce or eliminate restriction sites to make it more suitable for further manipulations.

After the vector comprising a DNA sequence coding for a plasminogen fragment of the invention is prepared, it is used to transform the host cells. Methods of transforming host cells are well known in the art, and any of these methods may be used.

Transformed host cells are selected in known ways and then cultured under conditions effective to produce the plasminogen fragment. The methods of culture are those well known in the art for the chosen host cell.

The expressed plasminogen fragment may be recovered using methods of recovering and purifying proteins from recombinant cell cultures which are well known in the art. In particular, antibodies which bind selectively to the plasminogen fragments of the invention may be used to purify the fragments (see below).

The invention also provides methods of treating an angiogenic disease. An angiogenic disease is one caused by, involving or dependent on angiogenesis. Angiogenic diseases include neoplastic diseases (e.g., tumors and tumor metastasis), benign tumors (e.g., hemangiomas, acoustic neuromas, neurofibromas, trachomas, pyrogenic granulomas), connective tissue disorders (e.g., rheumatoid arthritis and atherosclerosis), ocular angiogenic diseases (e.g., diabetic retinopathy, retinopathy of prematurity, macular degeneration, corneal graft rejection, neovascular glaucoma, retrolental fibroplasia, rubeosis), cardiovascular diseases, cerebral vascular diseases, diabetes-associated diseases and immune disorders (e.g., chronic inflammation and autoimmunity).

The angiogenic disease may be treated by administering an effective amount of native angiostatin or of another plasminogen fragment having the N-terminal sequence of plasmin and containing at least a portion of kringle 5. Native angiostatin is preferred for the reasons given above.

The angiogenic disease may also be treated by administering an amount of a sulfhydryl donor sufficient to cause conversion of plasmin to angiostatin. An effective amount of a plasminogen activator may also be administered to the animal to produce plasmin from plasminogen. The plasminogen or plasmin may be those found endogenously in the animal or effective amounts of plasminogen or plasmin are also administered to the animal.

In addition, the angiogenic disease may be treated by administering an amount of a plasminogen activator sufficient to cause conversion of plasminogen to plasmin. the plasminogen may be that found endogenously or an effective amount of plasminogen may also be administered to the animal. The plasmin is converted to angiostatin by a sulfhydryl donor. The sulfhydryl donor may be one found endogenously in the animal or an effective amounts of sulfhydryl donor can also be administered to the animal. One example of an endogenous free sulfhydryl donor which can facilitate conversion of plasmin to angiostatin is glutathione. Glutathione is a well-known endogenous chemical, present in normal and cancer tissues, which is released by the cells into the extracellular environment. In this extracellular environment, glutathione may serve as a free sulfhydryl donor to mediate conversion of plasmin to angiostatin. Melloni et al. demonstrated that the levels of glutathione in epithelial lining fluid (ELF), obtained by bronchoalveolar lavage, was significantly greater from patients with lung cancer (1,485.5+/−208 μM) when compared with smokers (544+/−97.6 μM) and nonsmokers (339.3+/−112 μM) without cancer (p<0.05) (Melloni et al., Am. J. Respir. Crit. Care Med, 154 (6 Pt 1): 1706-11, 1996.). Of note, as shown by Soff and colleagues, 100 μM levels of glutathione can serve as a cofactor for angiostatin generation, supporting the paradigm that endogenous levels of glutathione or other free sulfhydryl donors can be sufficient to convert endogenous plasmin to angiostatin (Gately et al., Proc. Natl. Acad. Sci. USA, 94 (20): 10868-72, 1997).

Any animal suffering from an angiogenic disease can be treated. Suitable animals treatable according to the invention include mammals, such as dogs, cats, horses, other domestic animals, and humans.

Effective dosage forms, modes of administration and dosage amounts for the various compounds for treating angiogenic diseases may be determined empirically, and making such determinations is within the skill of the art. It is understood by those skilled in the art that the dosage amount will vary with the activity of the particular compound employed, the severity of the angiogenic disease, the route of administration, the rate of excretion of the compound, the duration of the treatment, the identify of any other drugs being administered to the animal, the age, size and species of the animal, and like factors known in the medical and veterinary arts. In general, a suitable daily dose of a compound of the present invention will be that amount of the compound which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day.

The compounds of the present invention may be administered to an animal patient for therapy by any suitable route of administration, including orally, nasally, rectally, vaginally, parenterally (e.g., intravenously, intraspinally, intraperitoneally, subcutaneously, or intramuscularly), intracisternally, transdermally, intracranially, intracerebrally, and topically (including buccally and sublingually). The preferred routes of administration are subcutaneous, orally and intravenously. The use of biodegradable polymers similar to that described by Brem, et al., Lancet, 345, 1571 (1995) for the local sustained release of pharmacological agents following incorporation into the biodegradable polymers is also a preferred method of administration. Implantation of the drug-impregnated polymer at, e.g., a tumor site, allows prolonged local exposure with minimal systemic exposure.

While it is possible for a compound of the present invention to be administered alone, it is preferable to administer the compound as a pharmaceutical formulation (composition). The pharmaceutical compositions of the invention comprise a compound of the invention as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the patient.

Pharmaceutical formulations of the present invention include those suitable for oral, nasal, ophthalmic, topical, rectal, vaginal and/or parenteral administration. Regardless of the route of administration selected, the compounds of the present invention are formulated into pharmaceutically-acceptable dosage forms by conventional methods known to those of skill in the art.

The amount of active ingredient which will be combined with a carrier material to produce a single dosage form will vary depending upon the host being treated, the particular mode of administration and all of the other factors described above. The amount of active ingredient which will be combined with a carrier material to produce a single dosage form will generally be that amount of the compound which is the lowest dose effective to produce a therapeutic effect or the maximally-tolerated dose that yields a therapeutic increment for life-threatening illnesses, such as cancer.

Methods of preparing pharmaceutical formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound of the present invention as an active ingredient. A compound of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of a compound of the invention to the body. Such dosage forms can be made by dissolving, dispersing or otherwise incorporating a compound of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical compositions of this invention suitable for parenteral administrations comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

An angiogenic disease can also be treated by gene therapy. In particular, a transgene comprising DNA coding for a plasminogen fragment having the N-terminal sequence of plasmin and containing at least a portion of kringle 5 operatively linked to expression control sequences is administered to an animal suffering from such a disease. Preferably the plasminogen fragment coded for by the transgene is native angiostatin. The preparation of DNA coding for the plasminogen fragments of the invention, including native angiostatin, operatively linked to expression control sequences is described above. Expression of the transgene in the animal results in the production of the plasminogen fragment, which inhibits angiogenesis in the animal.

Methods and materials for gene therapy are well known in the art. See Culver, Gene Therapy: A Primer for Physicians (Revised 2nd ed., 1996), U.S. Pat. Nos. 5,521,291, 5,460,831 and 5,559,099, PCT applications WO 95/29242, WO 96/14876, and WO 96/35774, all of which are incorporated herein by reference in their entirety. See also, Kirshenbaum, et al., J. Clin. Invest., 92, 381-387 (1993) and Drazner et al., J. Clin. Invest., 99, 288-296 (1997). In particular, suitable methods and vehicles for delivery of transgenes are known and may be used to deliver the transgenes of the invention.

For instance, the transgene can be transfected into desired cells in vitro, and the transformed cells injected into an animal suffering from an angiogenic disease, preferably after expansion of the number of transformed cells. Methods of transfecting a transgene into cells in vitro are well known and include electroporation, direct injection of naked DNA into cells, particle bombardment, delivery by liposomes or other lipid-based carriers, delivery by viral vectors, etc.

Alternatively, the transgene can be administered to the animal in such a manner that it transforms cells within the animal. Methods of delivering transgenes in vivo are also well known and include direct injection of naked DNA into desired tissues, organs, or tumors, use of liposomes and other lipid-based carriers to deliver the transgene, use of a noninfectious viral vector (e.g., a replication-deficient adenoviral vector) to deliver the transgene, use of targeted vehicles (a vehicle that allows the vehicle to bind to, and deliver the transgene to, a specific cell, tissue, organ or tumor such as liposomes having a tumor-specific antibody attached to them) to deliver the transgene, etc.

The invention also provides antibodies which bind selectively to a plasminogen fragment of the invention, including antibodies which bind selectively to native angiostatin. “Binds selectively” means that the antibody binds to a plasminogen fragment of the invention, such as native angiostatin, in preference to plasminogen or plasmin.

Antibodies coming within the scope of the invention include polyclonal antibodies, affinity-purified antisera, monoclonal antibodies, fragments of antibodies (such as Fab, F(ab′) or F(ab′)₂) that are capable of binding antigen, any known isotype or subclass of antibody, and engineered antibodies (such a single-chain antibody prepared by recombinant DNA techniques). The only requirements are that the final antibody preparation have specificity is for the plasminogen fragment and be capable of binding selectively to the fragment.

Methods of making antibodies and fragments of antibodies are well known in the art.

For instance, the antibodies of the present invention may be prepared by injecting a suitable host animal (such as a rabbit, goat, horse or other mammal) with a plasminogen fragment of the invention in admixture with an adjuvant. The injections of the fragment are continued until an antiserum of suitable titer is obtained. The antiserum is harvested and may be further purified using known techniques if needed or desired. For instance, the antibodies may be affinity purified or may be fractioned such as by DE-52 chromatography.

Preferably, however, the antibodies of the invention are prepared by somatic cell hybridization by fusing cells from an immunized animal (such as rats, hamsters, mice or other mammal) with an immortal cell line such as myeloma cells. The fused cells are cloned, and monoclonal antibodies of appropriate specificity can be isolated by screening the cloned fused cells. Techniques of preparing monoclonal antibodies are well-known.

Antibodies which bind selectively to a plasminogen fragment of the invention can be used to purify the plasminogen fragments from fluids containing them. Such fluids include culture media, such as those resulting from practice of the methods of the invention for producing native angiostatin and the plasminogen fragments of the invention (see above). Native angiostatin would be found, in addition, in body fluids (e.g., blood, plasma, serum, saliva, urine and fluids produced by tumors).

To purify a plasminogen fragment, the fluid containing it is contacted with an antibody specific for the particular fragment. Preferably, the antibody is attached to a solid surface before being contacted with the fluid containing the fragment. Suitable solid surfaces are well known in the art and are available commercially. Examples include glass, polyacrylamide, polymethylmethacrylate, polycarbonate, polyacrylonitrile, polyethylene, polypropylene, polystyrene, latex beads, agarose beads, and nylon.

The antibody is preferably attached covalently to the solid surface. Methods and agents for attaching antibodies covalently to solid surfaces are well known in the art. Suitable agents include carbodiimide, cyanoborohydride, diimidoesters, periodate, alkylhalides, succinimides, dimethylpimelimidiate and dimaleimides. See Blair et al., J. Immunol. Methods, 59, 129 (1993); Blair et al., Cancer Res., 41, 2700 (1981); Gautheier et al., J. Expr. Med., 156, 766 (1982).

The specific concentrations of reactants, the temperature and time of incubation, as well as other conditions for obtaining binding of the antibody to the plasminogen fragment, can be varied depending on such factors as the concentration of the plasminogen fragment in the fluid, the nature of the fluid and the like. Those skilled in the art will be able to determine operative and optimal conditions while employing routine experimentation.

After the antibody has bound to the plasminogen fragment, the remainder of the fluid is separated from the bound plasminogen fragment. The plasminogen fragment is then released from the antibody by known methods.

Most preferably, a solid surface with the antibody attached to it is located in a column. For instance, a column filled with agarose beads having antibody attached to them. The fluid containing the plasminogen fragments is simply passed through the column, and the plasminogen fragments in the fluid bind to the antibody in the column and are retained in the column, while the remainder of the fluid passes through the column. After the column is washed, the plasminogen fragments are released from the antibody.

Antibodies of the invention which bind selectively to native angiostatin can also be used to detect or quantitate native angiostatin for the diagnosis of an angiogenic disease or to monitor for the recurrence of such a disease. Such antibodies can also be used to study the mechanism of action of angiostatin in the body.

Native angiostatin may be detected in materials such as body fluids (see above), cells and tissues (tumor tissue, placenta, uterus, brain, liver and intestines). Native angiostatin may be released from the cells or tissues by known extraction techniques, or intact cells or tissue sections may be used.

The native angiostatin in fluids or extracts can be detected or quantitated using conventional immunoassay techniques. Such techniques include agglutination, radioimmunoassay, enzyme immunoassays, fluorescence assays, colorimetric assays, etc. The immunoassay may be performed in the competitive binding format or may be an immunometric assay. It may be a homogenous or heterogenous assay. Suitable homogenous techniques are fluorescence quenching and enhancement, energy transfer immunoassay, double antibody steric hinderance immunoassay, substrate-labeled immunoassay, prosthetic group-labeled immunoassay and enzyme modulator-labeled immunoassay.

The native angiostatin on cells or tissues can be detected by standard immunohistochemical techniques well known in the art. For example, tumors are biopsied or collected, and tissue sections cut with a microtome to examine sites of native angiostatin production. Such information is useful for diagnostic and possibly therapeutic purposes in the detection and treatment of cancer and is useful for research purposes for studying the mode of action of angiostatin.

The native angiostatin can be detected or quantified using a labeled antibody which binds selectively to native angiostatin (primary antibody) or a labeled component that binds to immunoglobulin, such as another antibody (secondary antibody) or protein A. Suitable labels for either the primary antibody or for the component which binds to the primary antibody are well-known in the art. They include: 1) enzymes (e.g., horseradish peroxidase, malate dehydrogenase, staphylococcal nuclease, delta-5-steroid isomerase, yeast alcohol dehydrogenase, alpha-glycerophosphate dehydrogenase, triose phosphate isomerase, alkaline phosphatase, asparaginase, glucose oxidase, beta-galactosidase, ribonuclease, urease, catalase, glucose-6-phosphate dehydrogenase, glucoamylase and acetylcholine esterase); 2) fluorophores (such as fluorescein isothiocyanate, rhodamine, phycoerythrin, phycocyanin, allophycocyanin, o-phtaldehyde and fluorescamine), 3) radionucleotides (such as ¹²⁵I); 4) bioluminescent labels (such as luciferin, luciferase and aequorin); 5) chemiluminescent labels (such as luminol, isoluminol, aromatic acridinium ester, imidazole, acridinium salt and oxalate ester); 6) particulate lables (such as gold nanoparticles); and 7) biotin/avidin or biotin/streptavidin. The binding and detection of these labels can be performed using standard techniques known to those skilled in the art.

The specific concentrations of reactants, the temperature and time of incubation, as well as other conditions, can be varied in whatever immunoassay or immunohistochemical technique is employed, depending on such factors as the concentration of the native angiostatin in the sample, the nature of the sample and the like. Those skilled in the art will be able to determine operative and optimal conditions for each determination while employing routine experimentation.

A test kit for detecting or quantitating native angiostatin is also part of the invention. The kit is a packaged combination of one or more containers holding reagents useful in performing the immunoassays or immunohistochemical techniques of the invention. Suit-able containers for the reagents of the kit include bottles, vials, test tubes, microtiter plates, dipsticks, strips, and other solid surfaces.

The kit will comprise a container of an antibody which binds selectively to native angiostatin. These antibodies are those described above. The antibody may be in solution, may be lyophilized, or attached to a solid surface, and may be labeled or unlabeled. The solid surfaces are the types described above, and the antibody is attached as described above.

The kit may further comprise a container holding the above-described labeled component that binds to the primary antibody. The labels are those described above.

Finally, the kit may also contain other materials which are known in the art and which may be desirable from a commercial and user standpoint. Such materials may include a sample of native angiostatin (for standardizing immunoassays or for binding to cells or tissues in immunohistochemical techniques), buffers, enzyme substrates, diluents, and equipment for performing the immunoassay or immunohistochemical technique.

### EXAMPLES

### Example 1

### Preparation of Conditioned Medium Containing Plasminogen-Angiostatin Converting Activity (PACA)

This example demonstrates that a variety of cells express enzymatic activity that can generate bioactive angiostatin from purified human plasminogen or plasmin. Affinity-purified angiostatin generated by incubating plasminogen or plasmin with serum-free conditioned medium (SFCM) inhibited human endothelial cell proliferation, migration induced by angiogenic factor basic fibroblast growth factor (bFGF), endothelial cell tube formation, and bFGF-induced corneal angiogenesis. Serine proteinase inhibitors, but not inhibitors of metallo-, cysteine, or aspartic proteinases, blocked angiostatin generation. Elastatinal, a specific inhibitor of elastase, failed to block angiostatin generation, indicating that an elastase is not responsible for the conversion of plasminogen to angiostatin. Instead, the data show that serine proteinase activity is necessary for angiostatin generation.

### A. Methods

1. Cell Culture. The human umbilical vein endothelial cells (HUVEC), were grown in RPMI supplemented with 20% bovine calf serum (Hyclone Laboratories Inc., Logan Utah #A-2151-L), 100 U/ml penicillin G, 100 mg/ml streptomycin, L-glutamine, (Gibco BRL), 2500 U Sodium heparin (Fisher Scientific, Itasca, II), and 50 mg/ml endothelial cell growth supplement (Collaborative Biomedical Research, Bedford, Mass.). The other cells listed in Table 1 were grown in RPMI-1640 supplemented with 10% fetal bovine serum, 100 U/ml penicillin G, 100 mg/ml streptomycin (Gibco BRL, Gaithersburg, Md.). Cells were maintained at 37° C. in a humidified incubator in an atmosphere of 5% CO₂. To generate SFCM, confluent cell monolayers were washed twice with phosphate buffered saline, then serum-free RPMI was added. The next day the SFCM was collected and centrifuged at 3000 rpm for 15 minutes to remove insoluble cellular debris.

2. Angiostatin Generation. Two micrograms of human plasminogen, obtained by lysine-sepharose affinity chromatography of human plasma (Castellino & Powell, Methods Enzymol, 80, 365-78 (1981)), or human plasmin (#527624, Calbiochem-Novabiochem Corp., La Jolla, Calif.) were added to 100 μl aliquots of SFCM and the mixture incubated at 37° C. overnight. Aliquots were analyzed for angiostatin generation by western blot (see below). Plasminogen cleavage by SFCM was also assessed in the presence of proteinase inhibitors (Boehringer Mannheim, Indianapolis, Ind.).

3. Western Blot. Samples were electrophoresed under non-reducing conditions on 12% polyacrylamide gels (NOVEX, San Diego, Calif.), in Tris-Glycine running buffer (Laemmli, Nature, 227, 680-685 (1970)), and electrotransferred to a 0.45 μM polyvinylene difluoride (PVDF) membrane (Immobilon, Millipore, Bedford, M.A.). The membrane was then blocked for 30 minutes in blocking buffer (1% bovine serum albumin in Tris-buffered saline) and probed with a 1:1000 dilution of a monoclonal antibody to the kringles 1-3 (K1-3) fragment of human plasminogen (VAP 230L, Enzyme Research Laboratories, Inc., South Bend, Ind.). Following washing, the membrane was incubated for 30 minutes with an alkaline phosphatase conjugated goat anti-mouse IgG secondary antibody (Kirkegaard & Perry Laboratories (KPL), Gaithersburg, Md.) and developed using 5-bromo-4-chloro-3-indoyl-phosphate/nitroblue tetrazolium (KPL).

4. Zymographic Analysis. Zymograms to detect matrix metalloproteinase activity were performed as described previously. Heussen & Dowdle, Anal. Biochem., 102, 196-202 (1980).

5. Chromogenic Peptide Substrates. To determine if an elastase was present, 50 μl of SFCM were incubated with 0.3 mM of chromogenic peptide substrates specific for elastase (substrate I, MeOSuc-Ala-Ala-Pro-Val-pNA, SEQ ID NO:15; substrate II, Boc-Ala-Ala-Pro-Ala-pNA, SEQ ID NO:16); substrate III,pGlu-Pro-Val-pNA; substrate IV, Suc-Ala-Ala-Pro-Abu-pNA) (Calbiochem-Novabiochem Corp.), at 37° C. for 2-18 hours. Substrate cleavage was determined by monitoring the absorbance at 405 nm (Molecular Devices, Menlo Park, Calif.).

6. Lysine-Sepharose Purification of Angiostatin. To generate purified angiostatin for bioactivity analyses, human plasminogen was incubated with PC-3 SFCM at 20 μg/ml overnight at 37° C. The reaction product was applied to a lysine-sepharose column (Pharmacia Biotech), pre-equilibrated with TBS (50 mM Tris, pH 7.5, and 150 mM NaCl) Following washes with TBS to remove non-specifically bound protein, angiostatin was eluted in 0.2 M epsilon aminocaproic acid (EACA) in TBS. The eluted fraction was dialyzed (molecular weight cut off 12,000-14,000) to phosphate buffered saline. To remove residual plasmin, the angiostatin was applied to a soybean trypsin inhibitor agarose (Sigma Chemical Co., St. Louis, Mo.) column, and the flow-through collected, filter-sterilized and stored at −80° C. until used. Angiostatin was quantitated by measuring the absorbance at 280 nm, using an A^{1%}/1 cm of 8.0. Sottrup-Jensen et al., in Progress in Chemical Fibrinolysis and Thrombolysis, vol. 3, pages 191-209 (Davidson et al. eds. 1978). The purified angiostatin was also examined by Coomassie brilliant blue staining of polyacrylamide gels, and immunodetection by western blot. Elastase-generated angiostatin, purified from human plasma as described in O'Reilly, et al., Nature Med., 2, 689-692 (1996), was a generous gift from M. S. O'Reilly, Children's Hospital, Harvard University, Boston, Mass.

7. Microsequence Analysis of Angiostatin. To determine the NH₂-terminus of the angiostatin bands, 10 μg/ml of the affinity-purified angiostatin prepared by incubating plasminogen with PC-3 SFCM was electrophoresed on a 12% SDS-polyacrylamide gel, electroblotted to a PVDF membrane, and stained with Coomassie blue. The bands were excised, placed on Porton sample support discs, and sequenced using a pulse liquid-phase sequencer with phenylthiohydantoin analysis.

8. Endothelial Cell proliferation Assay. Cell proliferation was determined utilizing the CellTiter 96™ AQ Non-Radioactive Cell Proliferation Assay (Promega Corp., Madison, Wis.). The human endothelial cells were plated in a 96-well tissue culture plates (Becton Dickinson, Lincoln Park, N.J.) at a concentration of 5.0×10³ cells/well. The following day, 1, 5, 8, or 10 μg/ml of angiostatin in fresh medium was added to triplicate wells. Wells without angiostatin served as control. The cells were incubated for 72 hours, and an absorbance read at 490 nm, reflecting the number of proliferating cells, was measured using an automated microplate reader (Molecular Devices). The results are reported as the percent of non-treated control cell number.

9. Endothelial Cell Migration Assay. To determine the ability of angiostatin prepared by incubation of plasminogen with PC-3 SFCM to block migration of endothelial cells towards an angiogenic factor, bFGF, migration assays were performed in a modified Boyden chamber using bovine capillary endothelial cells (a kind gift of Dr. Folkman, Harvard Medical School, Boston, Mass.) as described previously. Dameron et al., Science, 265, 1582-84 (1994). Cells were grown in Dulbecco's modified Eagle's medium (DMEM) with 10% donor calf serum and 100 mg/ml endothelial cell mitogen and used at passage 15. To assess migration, the cells were serum starved overnight in DMEM supplemented with 0.1% bovine serum albumin (BSA), harvested, suspended in DMEM/BSA, plated at 10⁶/ml on the lower surface of a gelatinized membrane (Nucleopore Corp., Plesanton, Calif.) in an inverted Boyden chamber, and incubated for 1.5-2 hours to allow cell attachment. The chambers were reinverted, test material was added to the top well, and the chamber incubated for an additional 3-4 hours. Membranes were then fixed and stained and the number of cells that migrated to the top of the filter in 10 high-power fields was determined. DMEM with 0.1% BSA was used as a negative control, and bFGF (provided by Dr. Noel Bouck and prepared as described in Dameron et al., Science, 265, 1582-1584 (1994)) at 10 ng/ml was used as a positive control.

10. Endothelial Cell Tube Formation. HUVEC were plated on gels of Matrigel (kindly provided by Hynda Kleinman, National Institute of Dental Research) in 24-well tissue culture plates as described previously. Schnaper et al., J. Cell. Physiol, 156, 235-246 (1993). Angiostatin, prepared by incubation with PC-3 SFCM, in non-conditioned RPMI was added to the wells, followed by cells at a final concentration of 4.0×10⁴ cells in 1 ml of 50% HUVEC culture medium, 50% RPMI. Each angiostatin or control condition was assayed in triplicate. The cultures were incubated for 16-18 hours at 37° C., in a 5% CO₂ humidified atmosphere, then fixed with Diff-Quick Solution II (Baxter, McGraw Park, Ill.). A representative area of the tube network was photographed using a Polaroid MicroCam camera at a final magnification of 35X. The photographs were then quantitated by a blinded observer who measured the length of each tube, correcting for portions of tubes that were incomplete. The total length of the tubes was determined for each photograph and the mean tube length was determined. The results were expressed as the mean±standard error of the mean.

11. Corneal Angiogenesis Assay. The corneal assay was performed as described previously. Polverini et al., Methods Enzymol, 198, 440450 (1991). Briefly, 5 μl hydron pellets (Hydron Laboratories, New Brunswick, N.J.) containing 10 μg/ml bFGF or bFGF plus 1 or 10 μg/ml angiostatin were implanted into the cornea of anesthetized rats. After 7 days, the animals were sacrificed and corneal vessels were stained with colloidal carbon and corneas were examined for angiogenic activity.

### B. Results

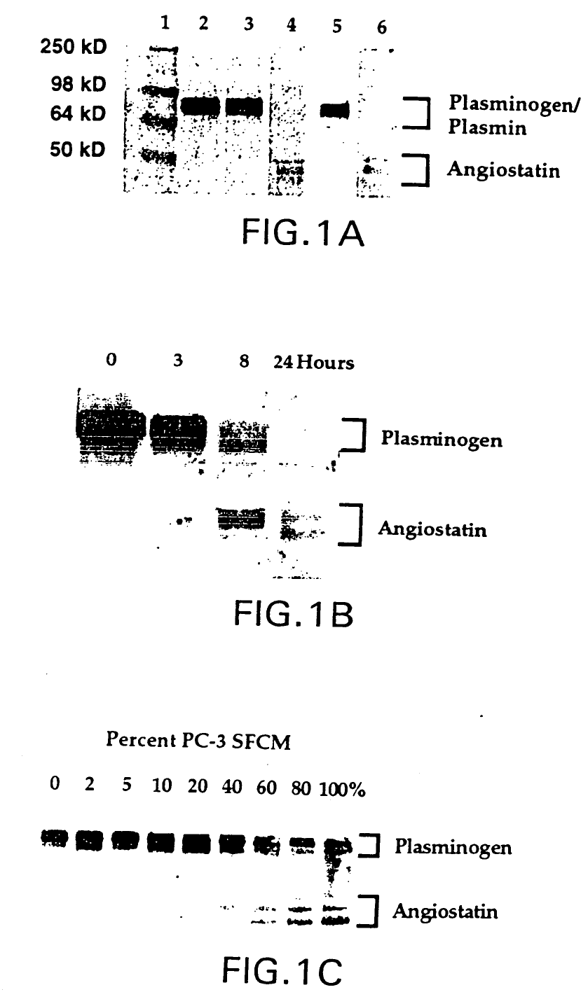

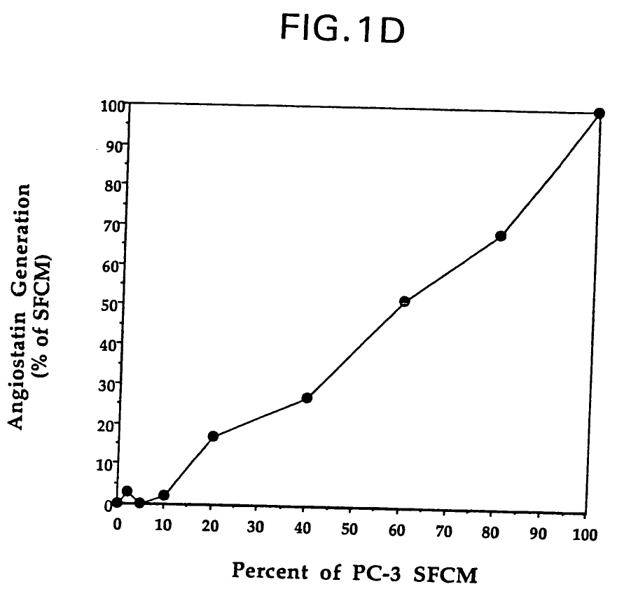

1. Angiostatin Generation BY Conditioned Culture Medium. Incubation of human plasminogen with the SFCM produced by PC-3 cells resulted in the generation of multiple immunoreactive bands at approximately 50 kD (FIG. 1A), similar to those observed by O'Reilly et al. Cell, 79,315-328 (1994). Examination of SFCM from additional cell lines also revealed the generation of the multiple bands, similar to the PC-3 SFCM (data not shown). These cell lines are listed in Table 1 below.

The initial indication that the product was angiostatin was based on the immunoreactivity with the monoclonal antibody specific for kringles 1-3 (K1-3) of plasminogen and the size of the cleavage product. Subsequent confirmation that the plasminogen cleavage product was bioactive angiostatin is described below.

Angiostatin generation by PC-3 SFCM was time-dependent. There was a significant decrease in the plasminogen substrate and a corresponding increase in angiostatin beginning at 3 hours, with complete conversion to angiostatin by 24 hours (FIG. 1B). Dilution of the PC-3 SFCM resulted in a proportional decrease in angiostatin generation (FIGS. 1C and 1D).

To determine whether plasmin, the activated form of the zymogen plasminogen, could also be converted to angiostatin, plasmin was evaluated as a potential substrate. Incubation of plasmin with PC-3 SFCM yielded a product indistinguishable from the plasminogen-derived angiostatin (FIG. 1A). In kinetic studies, plasmin was converted to angiostatin at a comparable rate to the plasminogen; 50% conversion by 8 hours, with complete conversion by 24 hours (data not shown). These data suggest that in vitro both plasminogen and plasmin are substrates from which angiostatin can be generated.

TABLE 1
Cell Lines Tested for Angiostatin-Generating Activity

| | Activity |
|---|---|
| Human Prostate Carcinoma | |
| PC-3 | +++ |
| DU-145 | ++ |
| Ln-CaP | + |
| Human Breast Carcinoma | |
| MDA-MB-231 | ++ |
| MCF-7 | +/− |
| Human Glioma | |
| U-373 | + |
| U-118 | + |
| A-172 | ++ |
| U-87 | + |
| Mouse Melanoma | |
| B16F10 | ++ |
| Bovine Smooth Muscle | |
| Primary cell line | ++ |
| Bovine Aortic Endothelial Cells | |
| BAEC | ++ |

2. Enzymatic Class Of Plasminogen-Angiostatin Converting Activity. To determine the proteolytic class of the angiostatin generating activity, PC-3 SFCM was incubated with plasminogen in the presence of various proteinase inhibitors. The proteinase inhibitors were added to the SFCM/plasminogen mix prior to the overnight incubation. Samples were analyzed by western blot for evidence of inhibition of angiostatin generation.

Only serine proteinase inhibitors blocked angiostatin generation (see Table 2 below). By contrast none of the other classes of proteinase inhibitors were effective.

Angiostatin can be generated in vitro by limited proteolysis of plasminogen by elastase. Sottrup-Jensen et al. in Progress in Chemical Fibrinolysis and Thrombolysis, 3, 191-209 (Davidson et al. eds. 1978); O'Reilly et al., Nature Med, 2, 689-692 (1996); Dong et al., Proc. Am. Assoc. Cancer Res., 37, 58 (1996). In the present study, angiostatin generation was not inhibited by elastatinal, a specific inhibitor of elastase (see Table 2 below). Additionally, no elastase activity was detected in PC-3 SFCM based on co-incubation of SFCM with 4 elastase-sensitive chromogenic substrates for 24 hours (data not shown). These data indicate that the human plasminogen-angiostatin converting activity is unlikely to depend on the action of an elastase. Furthermore, gelatin zymograms revealed no evidence of active or latent metalloproteinases in the PC-3 SFCM (not shown).

TABLE 2

| Proteinase | | | | Inhibitory |
|---|---|---|---|---|
| Inhibitor | Concentration | | Class | Activity * |
| Pefabloc | 4.0 | mM | Serine Proteinases | Complete |
| Aprotinin | 0.3 | μM | Serine Proteinases | Complete |
| Soybean Trypsin | 2.0 | mM | Serine Proteinases | Complete |
| Inhibitor | | | | |
| Benzamidine | 1-10 | mM | Serine Proteinases | Weak |
| Elastatinal | 50-100 | μM | Elastase | None |
| Antipain | 100 | μM | Limited Serine | None |
| dihydrochloride | | | Proteinases | |
| Leupeptin | 100 | μM | Serine and Thiol | None |
| | | | Proteinases | |
| Chymostatin | 100 | μM | Chymotrypsin | None |
| Bestatin | 10 | μM | Aminopeptidases | Weak |
| E-64 | 10 | μM | Cysteine Proteinases | None |
| Pepstatin | 1.0 | μM | Aspartic Proteinases | None |
| EDTA | 1-10 | mM | Metalloproteinases | None |
| 1-10 Phenanthroline | 10 | μM | Metalloproteinases | None |
| Phosphoramidon | 100 | μM | Metalloproteinases | None |

* Complete inhibition is defined as no immunoreactive angiostatin bands; weak inhibition results in the development of faint angiostatin immunoreactive bands; and none refers to the full generation of angiostatin.

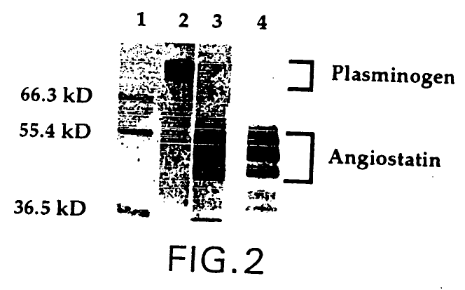

3. Purification Of Angiostatin. Angiostatin generated by PC-3 SFCM was affinity purified on lysine-sepharose (O'Reilly et al., Nature Med., 2, 689-692 (1996)), and the resulting product examined by western blot and Coomassie blue staining (FIG. 2). The amino-terminal sequence of all three bands was KVYLSECKTG [SEQ. ID NO:1] that corresponds to residues 78-87 of the plasminogen molecule, confirming that the product was an internal fragment of plasminogen.

4. Angiostatin Generated By PC-3 SFCM Inhibits Angiogenesis. Because angiogenesis represents a cascade of cellular processes that includes endothelial cell proliferation, migration, and tube formation, (Folkman & Shing, J. Biol. Chem, 267, 10931-10934 (1992)), multiple in vitro and in vivo assays related to angiogenesis were utilized to confirm that the product generated by incubating plasminogen with PC-3 SFCM was bioactive angiostatin.

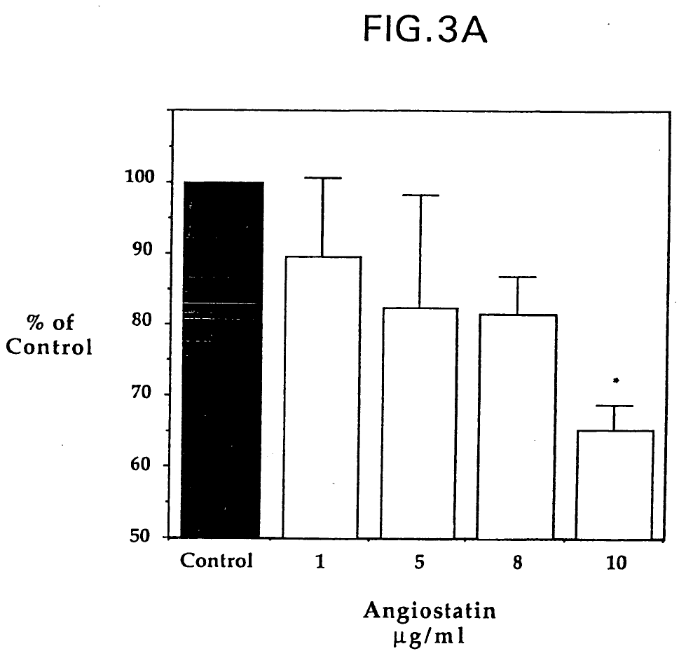

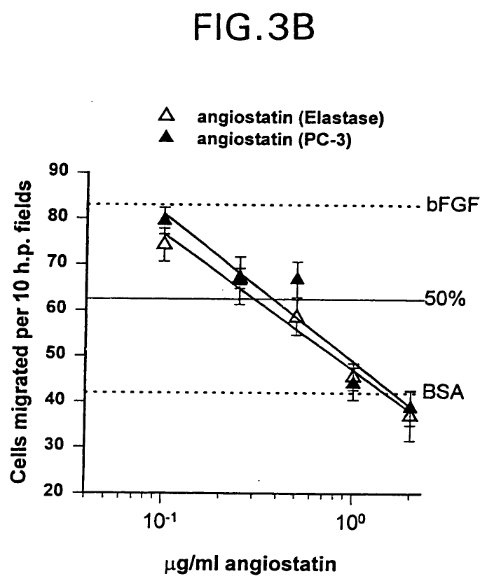

Affinity purified angiostatin generated by PC-3 SFCM inhibited human endothelial cell proliferation in a concentration-dependent manner, with significant inhibition observed at 10 μg/ml (P<0.05) in comparison to the non-treated control cell proliferation (FIG. 3A);

Angiostatin generated by PC-3 SFCM also inhibited the bFGF-induced migration of bovine capillary endothelial cells (FIG. 3B) with an ED₅₀ of 0.35 μg/ml. The dose/response curve of angiostatin generated by PC-3 SFCM was indistinguishable from that of elastase-generated angiostatin. Inhibition of migration occurred at a 10-fold lower concentration than required to inhibit proliferation, a finding that has been reported for other inhibitors of angiogenesis. Takano et al., Cancer Res., 54, 2654-2660 (1994). This may be due to the fact that the proliferation assay, in contrast to the migration assay, was conducted in RPMI supplemented with 20% calf serum and endothelial cell growth supplement, and therefore contained multiple stimulatory factors.

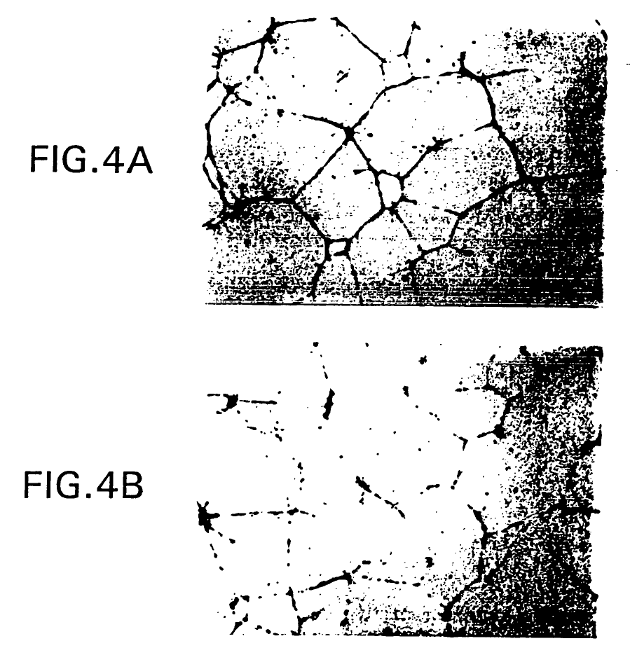

Endothelial cell tube formation on Matrigel was significantly inhibited at 15 μg/ml (FIGS. 4A and B); the mean length of tubes in non-treated control was 674.5±54 mm in comparison to angiostatin produced by PC-3 SFCM, 287.7±47 mm (P<0.005).

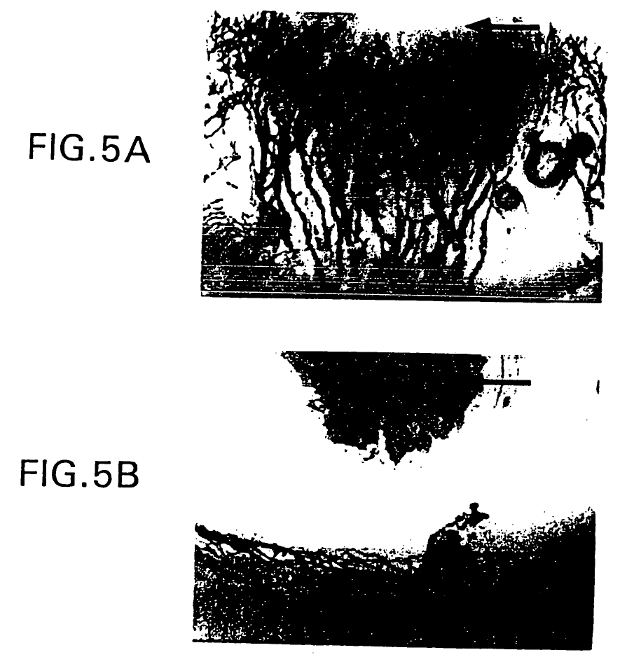

To determine the effect of angiostatin generated by PC-3 SFCM on corneal angiogenesis in vivo, its ability to block bFGF-induced angiogenesis in the corneal angiogenesis assay was tested. The bFGF pellet induced angiogenesis in 100% of implanted corneas (FIG. 5A). In contrast, angiostatin at 10 μg/ml completely inhibited the bFGF-induced angiogenic response in 3 of 3 animals (FIG. 5B). At a lower dose of 1.0 μg/ml, angiostatin completely blocked angiogenesis in 2 of 3 animals, with partial inhibition in the third animal.

Taken together, these data indicate that the angiostatin generated by the PC-3 SFCM is a potent inhibitor of both in vitro and in vivo angiogenesis.

### Example 2

### Identification of Factors Responsible For Converting Plasminogen to Angiostatin

The human prostate carcinoma cell line PC-3 was grown and PC-3 SFCM was prepared as described in Example 1. Angiostatin was generated by incubation with PC-3 SFCM or other materials identified below as described in Example 1. Western blots were performed as described in Example 1.

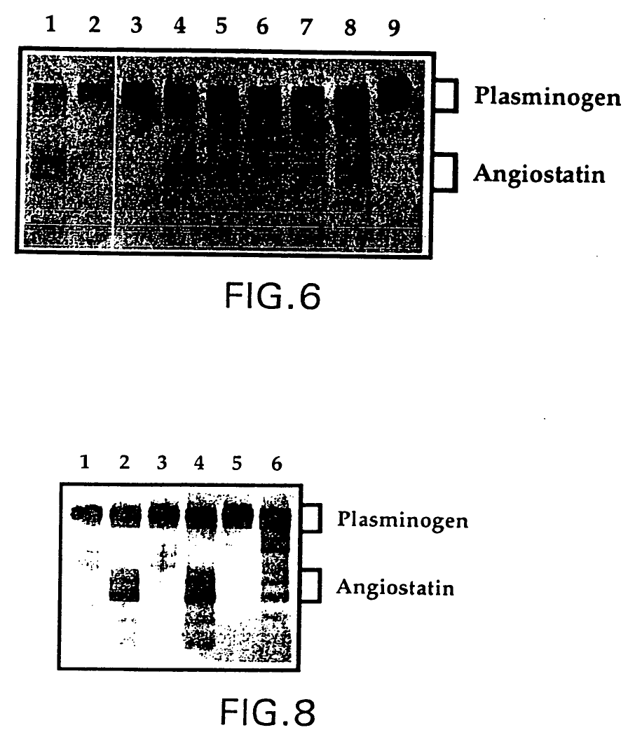

PC-3 SFCM was applied to a Reactive Red 120-Agarose column (Sigma Chemical Co.). The flow-through had no residual plasminogen-angiostatin generating activity (PACA) as demonstrated by western blot analysis (FIG. 6). The bound material was eluted with 1 M KCl according to the manufacturer's protocol, then dialyzed to Tris-buffered saline (TBS, 20 mM Tris, pH 7.4, 100 mM NaCl), with a molecular cut-off of 6000-8000 Dalton. PACA was not detected in the dialyzed fraction (FIG. 6). The observation that PACA was not detected in either the flow-through or the eluate led to the hypothesis that two or more factors are necessary to generate angiostatin from plasminogen or plasmin, and that the factors were separated by the Reactive Red 120-Agarose chromatography, with one or more factors being present in the elaute and one more factors being contained in the flow-through.

To test this hypothesis, the dialyzed eluate was recombined with the flow-through. The recombined materials were able to convert plasminogen into angiostatin. Supplementation of the eluate with fresh RPMI culture medium, as well as the Reactive Red 120-Agarose flow-through, restored the capacity of the eluate to generate angiostatin, suggesting that the necessary factor was a component of RPMI, and not a protein or other factor unique to the SFCM.

To further define the putative cofactor, the individual components of RPMI were evaluated for the ability to complement the Reactive Red 120-Agarose eluate. The cofactor was present in the RPMI amino acid mix (FIG. 6).

To determine which amino acid was capable of restoring PACA to the Reactive Red 120-Agarose eluate, the 20 amino acids found in RPMI were tested individually. L-cysteine was the only amino acid capable of restoring PACA to the Reactive Red 120-Agarose eluate (data not shown).

Because the addition of L-cysteine to the Reactive Red 120-Agarose eluate restored angiostatin generating activity, it was hypothesized that the cofactor was a sulfhydryl donor. Pharmacological reducing agents, D-penicillamine and captopril were therefore examined for the ability to restore PACA to the Reactive Red 12-agarose eluate. Addition of 100 μM D-penicillamine to the Reactive Red 120-Agarose eluate restored angiostatin generating activity. Captopril also restored angiostatin generating activity to the Reactive Red 120-Agarose eluate.

PC-3 SFCM was diluted to 50 mM Tris, pH 10.0,20 mM NaCl and applied to a Hi-Q Sepharose anion exchange resin (Bio Rad). No PACA was detected in the flow-through.

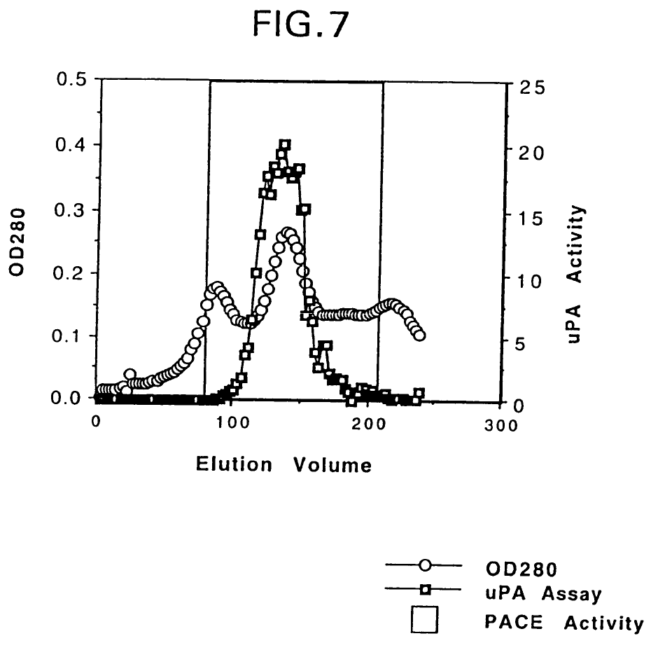

Preliminary experiments indicated that PACA eluted from the Hi-Q Sepharose column with 300 mM NaCl. Therefore, the bound material was eluted utilizing a linear gradient from 20 mM to 300 mM NaCl. PACA and urokinase-type plasminogen activator (u-PA) activity were measured in the fractions (after dilution to restore physiological NaCl concentrations). The u-PA activity and PACA co-purified (FIG. 7). Examination of the Reactive Red 120-Agarose eluate revealed it also contained u-PA.

As noted in Example 1, the NH₂-terminal cleavage of angiostatin is at Lys⁷⁷, a site that results from cleavage of Glu-plasminogen by plasmin. This suggests that plasmin generation may be a necessary intermediate step in angiostatin generation from plasminogen.

To determine if the factor in the Reactive Red 120-Agarose eluate was u-PA, u-PA was tested as a substitute for the Reactive Red 120-Agarose eluate. As illustrated in FIG. 8, u-PA was capable of generating angiostatin in the presence of boiled Reactive Red 120-Agarose flow-through or RPMI, both sources of sulfhydryl donors. This indicates that the only protein necessary for conversion of plasminogen to angiostatin is u-PA.

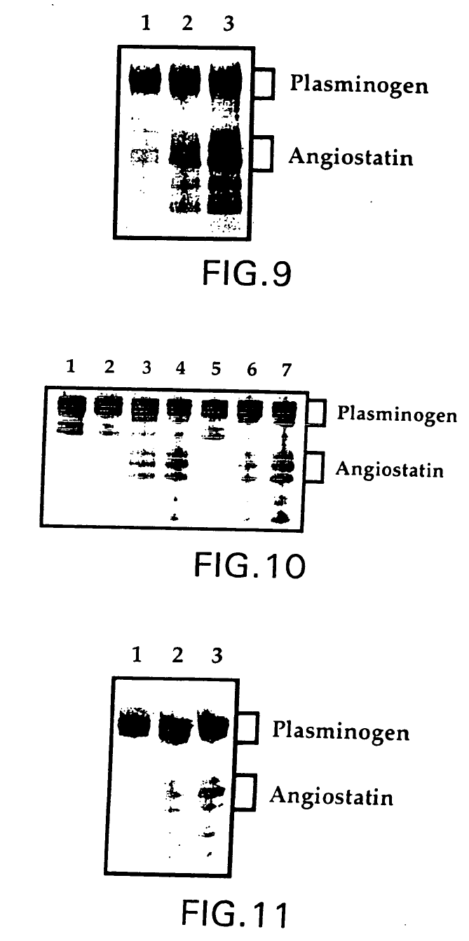

Next, u-PA, tissue-type plasminogen activator (t-PA), and streptokinase were tested in combination with the Reactive Red 120-Agarose flow-through for PACA. The plasminogen activators alone failed to generate angiostatin from plasminogen but, in the presence of the flow-through, angiostatin was produced (FIG. 9). These data suggest that plasmin generation is an intermediate for angiostatin generation, and that angiostatin generation is not dependent on which plasminogen activator is present.

### Example 3

### Generation of Angiostatin Using Plasminogen Activators and Sulfhydryl Donors

Having demonstrated that the only protein necessary for conversion of plasminogen to angiostatin is a plasminogen activator and that a sulfhydryl donor is a necessary cofactor, it was next determined if these components are sufficient for angiostatin generation. All incubations were performed at 37° C. for 18 hours in TBS, and the resulting samples were analyzed for angiostatin by Western blot (performed as described in Example 1).

Incubation of u-PA with plasminogen and at least 5 μM reduced glutathione produced angiostatin (FIG. 10). No angiostatin was produced in the absence of the glutathione.

Use of 100 μM or 1 mM D-penicillamine in combination with u-PA was also capable of generating angiostatin (FIG. 11).

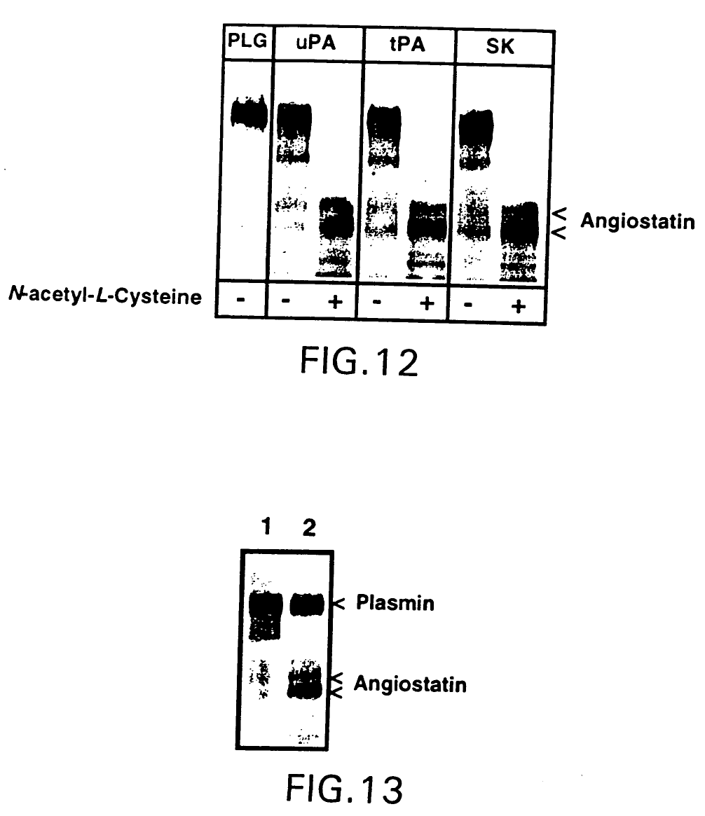

Finally, incubation of plasminogen (0.2 μM) with u-PA (0.2 nM), t-PA (1.0 nM), or streptokinase (8.0 nM) with 100 μM N-acetyl-L-cysteine resulted in the production of angiostatin (FIG. 12). Plasminogen was not converted to angiostatin in the absence of N-acetyl-L-cysteine.

These data show that plasminogen is converted to angiostatin by each of the classic plasminogen activators in the presence of a sulfhydryl donor, but not in the absence of the sulfhydryl donor. Further, these data and the data in Example 2 demonstrate that angiostatin is produced in the presence of physiological (L-cysteine, reduced glutathione) and pharmacological (captopril, D-penicillamine, N-acetyl-L-cysteine) reducing agents.

### Example 4

### Use of Plasmin for Angiostatin Generation

Two micrograms of human plasminogen in 100 μl of TBS was incubated with 10 μl of uPA-Sepharose (Calbiochem, La Jolla, Calif.) for 2 hours at 37° C. Following this incubation, the sample was centrifuged to sediment the uPA-Sepharose, and the supernatant containing plasmin was collected. The complete conversion of plasminogen to plasmin was confirmed by analysis of the supernatant on Coomassie-stained reduced polyacrylamide gels. The purified plasmin was then incubated for 18 hours at 37° C. with 100 μM N-acetyl-L-cysteine, and samples analyzed for angiostatin generation by Western blot (performed as described in Example 1).

The results are shown in FIG. 13. These results demonstrate that plasmin is a necessary intermediate in the generation of angiostatin from plasminogen, and that angiostatin can be produced by incubation of purified plasmin with a sulfhydryl donor.

### Example 5

### Treatment of Tumors in vivo with Sulfhydryl Donor, with and without Plasminogen Activator

Eleven female beige nude mice (Taconic Labs, Germantown, N.Y.) 6-8 weeks of age were injected subcutaneously in the right flank with 1.0×10⁶ murine hemangioendothelioma (EOMA) cells (generously provided by Dr. Robert Auerbach, Madison, Wis.) in 100 μl phosphate-buffered saline. The EOMA tumor cells were grown in Dulbecco's Modified Eagle Medium (DMEM) supplemented with 10% fetal bovine serum (FBS), 100 units/ml penicillin G, and 100 mg/ml streptomycin (Life Technologies Inc., Gaithersburg, Md.) and maintained at 37° C. in a humidified incubator in an atmosphere of 10% CO₂. The day of injection of the tumor cells was designated day 0.

Beginning on day 1, each of the mice was injected subcutaneously twice a day with the following:

TABLE 3

| Group And Number Of Mice | Treatment |
|---|---|
| Control (5) | Saline |
| NAC (4) | N-acetyl-L-cysteine in saline |
| | (6 mg per injection) |
| uPA + NAC(4) | urokinase-type plasminogen activator in |
| | saline (250 Units per injection) + |
| | N-acetyl-L-cysteine in saline |
| | (6 mg per injection) |

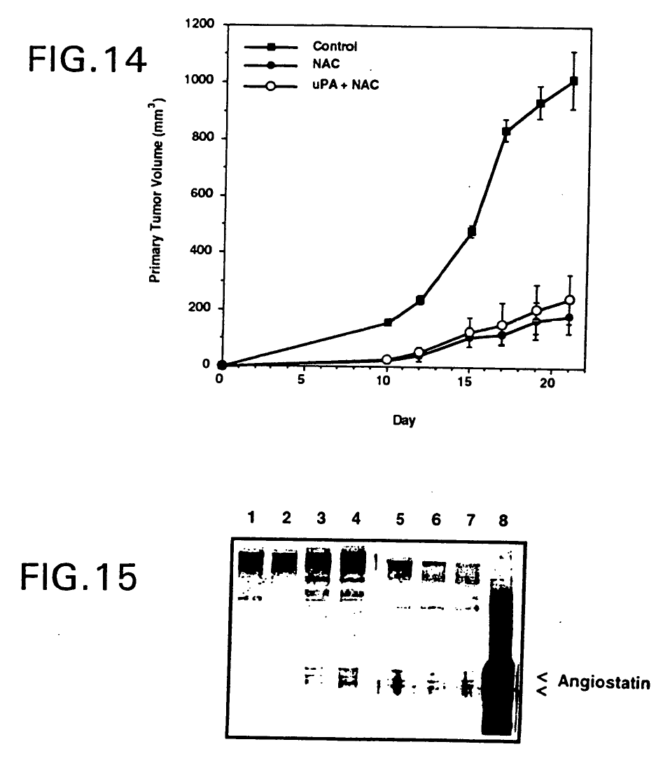

The size of the primary tumor in each mouse was measured three times weekly using tissue calipers, and tumor volume was determined using the formula (width²×length×0.52) (O'Reilly et al., Nature Medicine, 2, 689-692 (1996)). The results are presented in FIG. 14. As can be seen both treatment with NAC and treatment with uPA+NAC effectively and significantly decreased the mean tumor size as compared to the control group. It should be noted that one control mouse died on day 10 and another control mouse died on day 17. None of the mice treated with NAC or uPA+NAC died during the 21-day duration of the experiment.

Plasma samples taken from two of the control mice and three of the NAC-treated mice on the day of sacrifice were assayed for angiostatin by Western blot (performed as described in Example 1). As a control, two mice were injected subcutaneously with 1.00 mg of affinity-purified, cell-free angiostatin twice a day beginning on day 1 until 24 hours prior to sacrifice. Affinity-purified, cell-free angiostatin was generated as described in Example 3 and affinity purified on a lysine-sepharose column as described in Example 1. The results are shown in FIG. 15. As can be seen, administration of NAC to the mice caused the production of angiostatin in vivo (Lanes 5, 6 and 7). No angiostatin production was detected in control mice (Lanes 1 and 2).

### Example 6

### Determination of the C-Terminal Sequence of Native Angiostatin

Native angiostatin was generated by incubating human plasminogen (0.2 μM) with recombinant human u-PA (0.2 nM) (Abbott Laboratories, North Chicago, Ill.) and 100 μM N-acetyl-L-cysteine at 37° C. overnight. The material was then applied to a lysine-sepharose column (see Example 1), and the flow-through material was collected and concentrated. Aliquots of the concentrated flow-through material were electropheresed under non-reducing conditions on 12% polyacrylamide gels (NOVEX, San Diego, Calif.) in Tris-Glycine running buffer, electrotransferred to a 0.45 μm polyvinylene difluoride (PVDF) membrane (Immobilon, Millipore, Bedford, Mass.), and proteins stained with Coomassie blue.

The stained membrane showed two very prominent bands from the flow-through at approximately 30 kD. Although other bands were observed, the staining of these bands was considerably less than the staining of the two 30 kD bands, indicating that the two 30 kD bands contained the predominant constituents of the flow-through.

The N-terminal sequences of the proteins in the two 30 kD bands were determined by microsequence analysis as described in Example 1. The N-terminal sequence of the most prominent of the two bands was Lys Leu Tyr Asp Tyr Cys Asp Val [SEQ ID NO:2], while the sequence of the other band was Leu Tyr Asp Tyr Cys Asp Val [SEQ ID NO:3]. The location of these sequences in kringle 5 of plasminogen (see FIG. 16) and the prominence of the two bands provided extremely strong evidence that these were the fragments released as a result of the cleavage of plasminogen to form the C-terminal of native angiostatin.

From the N-terminal sequences of the two 30 kD fragments, it was deduced that the C-terminal sequence of the native angiostatin was Cys Tyr Thr Thr Asn Pro Arg [SEQ ID NO:4] or Cys Tyr Thr Thr Asn Pro Arg Lys [SEQ ID NO:5]. These C-terminal sequences would be formed by cleavage after amino acid 529 (Arg) or 530 (Lys) of human plasminogen, which is in kringle 5 (see FIG. 16), which are known plasmin cleavage sites. Cleavage at this point would give a plasminogen fragment of about the molecular weight observed for native angiostatin on polyacrylamide gel electrophoresis under non-reducing conditions (50-60 kD).

The N-terminal sequence of human native angiostatin [SEQ ID NO:1] is given above in Example 1. Thus, human native angiostatin was deduced to be a plasminogen fragment including most of kringle 5 with the N-terminal sequence

[SEQ ID NO:1]

Lys Val Tyr Leu Ser Glu Cys Lys Thr Gly

and the C-terminal sequence

[SEQ ID NO:4]

Cys Tyr Thr Thr Asn Pro Arg
or

[SEQ ID NO:5]

Cys Tyr Thr Thr Asn Pro Arg Lys.

### Example 7

### Antibody to Native Angiostatin

An antibody to the C-terminal of native human angiostatin (see Example 6) was prepared by immunizing three New Zealand white rabbits with a peptide having the sequence:

[SEQ ID NO:17]

Arg Asn Pro Asp Gly Asp Val Gly Gly
Pro Trp Cys Tyr Thr Thr Asn Pro Arg

(purified by RP-HPLC to >95% purity; Multiple Peptide Systems, San Diego, Calif.) conjugated 1:1 (weight/weight) to Keyhole Limpet Hemocyanin (KLH) using gluteraldehyde as the cross-linking agent. The rabbits were immunized with the peptide-KLH conjugate, suspended in phosphate buffered saline and emulsified by mixing with an equal volume of complete Freund's Adjuvant, injected into four subcutaneous dorsal sites. Subsequent immunizations were performed using incomplete Freund's Adjuvant. The rabbits were bled from the auricular artery, the blood allowed to clot, and the serum collected by centrifugation. Affinity purification was carried out using the synthetic peptide [SEQ ID NO:17] coupled to agarose. Crude serum was applied to the resin and, after washes, the adsorbed protein was eluted with a low pH glycine-HCl buffer. The antiserum recognized reduced and non-reduced plasminogen, plasmin and native angiostatin produced as described in Example 6, but not elastase-derived angiostatin (consisting of kringles 1-3).

### Example 8

### Treatment of Human Cancer Patients

Four human cancer patients were treated with a combination of a sulfhydryl donor and a plasminogen activator. Captopril was used as the sulfhydryl donor. Urokinase (uPA) or tissue plasminogen activator (tPA) was used as the plasminogen activator. The compounds administered and their doses and schedules of administration are summarized in Table 4 below. In addition, one of the patients (Case #2) received a single treatment with uPA alone (i.e., without the captopril).

Blood samples, in sodium citrate anticoagulant, were taken before, and at various times after, treatment was begun. Platelet-poor plasma was isolated from the blood, and stored frozen until tested.

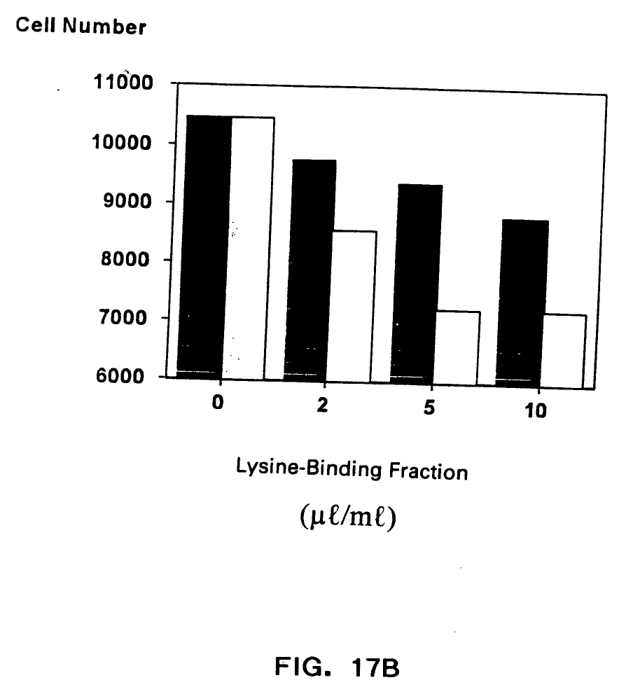

Western blots were performed on the plasma samples (diluted 1:20) in Tris-Buffered saline (20 mM Tris, pH 8.0, 150 mM NaCl) as described in Example 1. The results of the Western blots performed on some of the plasma samples taken from Case #2 are shown in FIG. 17A. As noted above, this patient received uPA alone and in combination with captopril. As can be seen in FIG. 17A, a marked increase in angiostatin levels was observed as a result of either treatment. This indicates that captopril, or other sulfhydryl donors, may not be necessary to convert plasmin to angiostatin in some situations. Such situations may include the presence of mesothelioma or other cancers that can release endogenous sulfhydryl donors or that, in some other way, mediate conversion of plasmin to angiostatin.

The lysine-binding proteins were purified from platelet-poor plasma samples obtained after the administration of uPA alone or uPA plus captopril to Case #2. First, 1 ml of platelet-poor plasma was diluted 1:40 with 20 mM Tris, pH 8.0, and incubated with 8 ml of Lysine-Sepharose (pre-equilibrated in 20 mM Tris, pH 8.0) for >12 hours with gentle shaking at 4° C. (In preliminary experiments, the 8:1 ratio of resin:plasma bound >99% of angiostatin and plasma). The plasma supernatant was separated by gentle centrifugation (2000×g), and the resin was washed 3 times with 40 ml of 100 mM NaCl, 20 mM Tris, 5 mM EDTA, pH 8.0. After washing and repeat centrifugation, the bound proteins were eluted with 4 ml of 200 mM EACA, 20 mM Tris, 5 mM EDTA, pH 8.0, and the elution dialyzed to 40 mM NaCl, 20 mM Hepes, 5 mM EDTA, pH 8.0, and spin concentrated (MW cutoff=10,000) to 500 microliters. On Western blot and Coomasie-stained polyacrylamide gel electrophoresis, the lysine-binding fractions contained plasminogen, angiostatin, and complexed angiostatin only.

To evaluate the antiangiogenic activity of the lysine-binding fractions of the plasma samples taken from Case #2, a cellular proliferation assay was performed as follows. Bovine aortic endothelial cells were plated in 24-well culture dishes at 1.0×10⁴ cells/well in DMEM supplemented with 2.5% heat-inactivated calf serum, 100 Units/ml penicillin G, 100 mg/ml streptomycin, and the cells were incubated overnight at 37° C. in a humidified incubator. On each of the following three days, fresh medium containing 3 ng/ml human bFGF (R&D Systems, Minneapolis, Minn.) alone or with various amounts of the lysine-binding fractions was added. As a positive control, 100 nM affinity-purified, cell-free angiostatin (produced as described in Example 6) was used. After 72 hours of treatment, cells were washed with phosphate buffered saline, dispersed with trypsin-EDTA, and the cell number was determined by counting from duplicate wells using a Coulter counter. The results are shown in FIG. 17B. As shown in that figure, administration of urokinase alone induced antiangiogenic activity as measured by this endothelial cell proliferation assay. While more antiangiogenic activity was induced by the administration of the combination of captopril and urokinase, the urokinase alone induced antiangiogenic activity. These data indicate that antiangiogenic activity is induced by plasminogen activators alone, although not as potently as by the plasminogen activators and free sulfhydryl donors in combination.

Case #1 was a 14 year old girl with recurrent Ewing's sarcoma of the left pelvis. After she could not tolerate chemotherapy or radiation therapy due to her extensive prior treatment, she received multiple cycles of the combination of captopril and urokinase. The doses and schedules of administration are summarized in Table 4. For the first several months, she received the captopril-urokinase combination for 3 consecutive days every 2 weeks. Subsequently, she received the combination 2 consecutive days every 3 weeks for a total of one year. A “cycle” refers to the 2 or 3 days of treatment, plus the days off treatment, until the therapy was begun again 2 or 3 weeks later. The Western blots revealed generation of angiostatin-related protein, which included free angiostatin as well as a complex of angiostatin with another protein or proteins. The other protein(s) has(have) not yet been identified. The large immunoreactive bands observed on the Western blot are believed to contain angiostatin because: (a) they cross-react with several antibodies to angiostatin, including kringle-dependent antibodies and COOH-terminus specific antibodies; (b) when affinity purified with lysine-Sepharose, and disulfide reduced, the complex yields monomeric angiostatin; and (c) the complex can be affinity purified with a resin comprising monoclonal antibodies to angiostatin coupled to Sepharose. Over three months of treatment, the patient achieved a complete remission. The therapy was continued for a full year, and the patient has remained in remission during the treatment period, as well as for 6 months after completion of treatment.

All of the cases, their treatments, and the results achieved are summarized in Table 4.

| | | Angiostatic | | | | |
|---|---|---|---|---|---|---|
| | | Cocktail (Drugs | Plasminogen | Captopril | | |
| Case | Disease | Administered) | Activator Doses | Doses | Schedule | Response |
| #1 | Recurrent | uPA/Captopril | uPA, 500 U/kg bolus, | 0.3 mg/kg/dose × | 3 Consecutive Days | Complete Remission |
| | Ewing's | | 200 U/kg/hr for 6 hrs. | 2 doses per day | Every 3 Weeks | |
| | sarcoma | | | of treatment | | |
| #2 | Mesothelioma | uPA/Captopril | uPA, intravenous (IV) | 18.75 mg/dose × | 3 Consecutive Days | >80% Tumor |
| | | | bolus, 500 U/kg | 2 doses per day | Every 2 Weeks | Regression |
| | | | uPA, continuous infusion, | of treatment | | |
| | | | 200 U/kg/hr for 6 hr. | | | |
| #3 | Ovarian | tPA/Captopril | tPA, IV bolus, 7 mg | 25 mg/dose × | 3 Consecutive Days | >50% Tumor |
| | Cancer | | tPA, continuous infusion, | 2 doses per day | Every 2 Weeks | Regression |
| | (Stage 4) | | 3 mg/hr for 6 hr. | of treatment | | |
| #4 5 | Hormone- | tPA/Captopril | tPA, IV bolus, 7.5 mg | 25 mg/dose × | Week 1; 5 consecutive days | >90% Decrease in |
| | Refractory, | | tPA, continuous infusion, | 2 doses per day | Week 2; 3 consecutive days | prostate-specific |
| | Metastatic | | 5 mg/hr for 6 hr. | of treatment | Week 3: 3 days, every other | antigen |
| | Prostate | | | | day | |
| | Cancer | | | | Week 4: 4 consecutive days | |
| | | | | | Week 5; 1 day | |

We claim:

1. A method of generating angiostatin in vitro comprising contacting plasminogen with a plasminogen activator and a sulfhydryl donor.

2. The method of claim 1 wherein the plasminogen activator is selected from the group consisting of urokinase, streptokinase, and tissue plasminogen activator.

3. The method of claim 1 wherein the sulfhydryl donor is selected from the group consisting of cysteine, N-acetyl cysteine, captopril, D-penicillamine, and reduced glutathione.

4. The method of claim 1 wherein the angiostatin is at least partially purified from the reaction mixture.

5. The method of claim 1 further comprising administering an effective amount of the angiostatin to an animal in need thereof.

6. The method of claim 4 further comprising administering an effective amount of the angiostatin to an animal in need thereof.

7. A method of generating angiostatin in vitro comprising:
contacting plasminogen with a plasminogen activator to produce plasmin; and
contacting the plasmin with a sulfhydryl donor to produce the angiostatin.

8. The method of claim 7 wherein the plasminogen activator is selected from the group consisting of urokinase, streptokinase, and tissue plasminogen activator.

9. The method of claim 7 wherein the sulfhydryl donor is selected from the group consisting of cysteine, N-acetyl cysteine, captopril, D-penicillamine, and reduced glutathione.

10. The method of claim 7 wherein the plasmin is at least partially purified prior to contacting it with the sulfhydryl donor.

11. The method of claim 7 wherein the angiostatin is at least partially purified from the reaction mixture.

12. The method of claim 7 further comprising administering an effective amount of the angiostatin to an animal in need thereof.

13. The method of claim 11 further comprising administering an effective amount of the angiostatin to an animal in need thereof.

14. A method of generating angiostatin in vitro comprising contacting plasmin with a sulfhydryl donor.

15. The method of claim 14 wherein the sulfhydryl donor is selected from the group consisting of cysteine, N-acetyl cysteine, captopril, D-penicillamine, and reduced glutathione.

16. The method of claim 14 wherein the angiostatin is at least partially purified from the reaction mixture.

17. The method of claim 14 further comprising administering an effective amount of the angiostatin to an animal in need thereof.

18. The method of claim 16 further comprising administering an effective amount of the angiostatin to an animal in need thereof.

19. A method of treating an angiogenic disease comprising administering to an animal suffering from such a disease an amount of a sulfhydryl donor effective to cause the conversion plasmin to angiostatin.

20. The method of claim 19 wherein the sulfhydryl donor is selected from the group consisting of cysteine, N-acetyl cysteine, captopril, D-penicillamine and reduced glutathione.

21. The method of claim 19 wherein an effective amount of plasmin is also administered to the animal.

22. The method of claim 19 further comprising administering an effective amount of a plasminogen activator to the animal to convert plasminogen to plasmin.

23. The method of claim 22 wherein the plasminogen activator is selected from the group consisting of urokinase, streptokinase and tissue plasminogen activator.

24. The method of claim 22 wherein an effective amount of plasminogen is also administered to the animal.

25. A composition for generating angiostatin comprising a sulfhydryl donor and a plasminogen activator.

26. The composition of claim 25 wherein the sulfhydryl donor is selected from the group consisting of cysteine, N-acetyl cysteine, captopril, D-penicillamine and reduced glutathione.

27. The composition of claim 25 wherein the plasminogen activator is selected from the group consisting of urokinase, streptokinase and tissue plasminogen activator.

28. The composition of claim 25 which is a conditioned culture medium produced by culturing cells capable of producing plasminogen activator in a culture medium or is a lysate of such cells.

29. A container holding a plasminogen activator, said container having a label thereon instructing administration of the plasminogen activator to an animal suffering from an angiogenic disease.

30. The container of claim 29 further holding a sulfhydryl donor and said label on said container instructing administration of the combination of the sulfhydryl donor and plasminogen activator to an animal suffering from an angiogenic disease.

31. A container holding a sulfhydryl donor, said container having a label thereon instructing administration of the sulfhydryl donor to an animal suffering from an angiogenic disease in an amount effective to cause conversion of plasmin to angiostatin.

32. A method of generating angiostatin comprising:
culturing cells capable of producing plasminogen activator in a culture medium for a time sufficient to produce conditioned culture medium (CCM) capable of converting plasminogen into angiostatin; and
contacting the CCM with plasminogen to produce the angiostatin.

33. The method of claim 32 wherein the cells are selected from the group consisting of cancer cells, primary endothelial cells, smooth muscle cells and fibroblasts.

34. The method of claim 32 wherein the angiostatin is at least partially purified from the CCM.

35. The method of claim 32 further comprising administering the angiostatin to an animal in need thereof.

36. The method of claim 34 further comprising administering the angiostatin to an animal in need thereof.

37. A method of generating angiostatin comprising:
culturing and thereafter lysing cells capable of producing plasminogen activator; and
contacting the lysate with plasminogen to produce the angiostatin.

38. A protein having the following characteristics:
(a) it is a fragment of plasminogen;
(b) its N-terminal amino acid is the same as the N-terminal amino acid of plasmin;
(c) its C-terminal amino acid is in kringle 5; and
(d) it inhibits angiogenesis.

39. The protein of claim 38 which comprises at least 50% of kringle 5.

40. The protein of claim 39 which comprises at least 75% of kringle 5.

41. The protein of claim 38 which is a fragment of human plasminogen and which has the following additional characteristic:
(e) it has an approximate molecular weight of 50-60 kD on polyacrylamide gel electropheresis under non-reducing conditions.

42. The protein of claim 41 having the following additional characteristics:
(f) it has the N-terminal sequence:
[SEQ ID NO:1]
Lys Val Tyr Leu Ser Glu Cys Lys Thr Gly;
and
(g) it has the C-terminal sequence:
[SEQ ID NO:4]
Cys Tyr Thr Thr Asn Pro Arg;
or
[SEQ ID NO:5]
Cys Tyr Thr Thr Asn Pro Arg Lys.

43. A DNA molecule comprising a sequence which codes for the protein of any one of claims 3842.

44. The DNA molecule of claim 43 wherein the coding sequence is operatively linked to expression control sequences.

45. A host cell comprising the DNA molecule of claim 44.

46. A method of producing a plasminogen fragment which inhibits angiogenesis comprising culturing the host cell of claim 45.

47. An antibody which binds selectively to native angiostatin.

48. A method of detecting or quantitating native angiostatin in a material suspected of containing native angiostatin, the method comprising:
contacting the material with the antibody of claim 47; and
detecting or quantitating any native angiostatin present in the material.

49. A kit for detecting or quantitating native angiostatin comprising a container holding the antibody of claim 47.

50. An antibody which binds selectively to the protein of claim 38.

51. A method of purifying a protein of claim 38 from a material containing it, the method comprising:
contacting the material with the antibody of claim 50 so that the antibody binds to the protein; and
separating the protein bound to the antibody from the remainder of the material.

52. A method of treating an angiogenic disease comprising administering to an animal suffering from such a disease an effective amount of the protein of any one of claims 38-42.

53. The method of claim 52 wherein the protein is native angiostatin.

54. A method of treating an angiogenic disease comprising administering to an animal suffering from such a disease a transgene comprising DNA coding for the protein of claim 38 operatively linked to expression control sequences.

55. The method of claim 54 wherein the protein coded for by the transgene is native angiostatin.

56. A method of treating an angiogenic disease comprising administering to an animal suffering from such a disease an amount of a plasminogen activator effective to cause the conversion plasminogen to plasmin.

57. The method of claim 56 wherein an effective amount of plasminogen is also administered to the animal.

58. The method of claim 56 wherein the plasminogen activator is selected from the group consisting of urokinase, streptokinase and tissue plasminogen activator.


SEQUENCE LISTING (17 sequences)

SEQ ID NO: 1 - protein, 10 aa, Homo sapiens
KVYLSECKTG

SEQ ID NO: 2 - protein, 8 aa, Homo sapiens
KLYDYCDV

SEQ ID NO: 3 - protein, 7 aa, Homo sapiens
LYDYCDV

SEQ ID NO: 4 - protein, 7 aa, Homo sapiens
CYTTNPR

SEQ ID NO: 5 - protein, 8 aa, Homo sapiens
CYTTNPRK

SEQ ID NO: 6 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 7 - protein, 812 aa, Bos taurus
MLPASPKMEHKAVVFLLLLFLKSGLGDLLDDYVNTQGASLLSLSRKNLAGRSVEDCAAKCEEETDFVCRAFQYHSKEQQCVVMAENSKNTPVFRMRDVILYEKRIYLLECKTGNGQTYRGTTAETKSGVTCQKWSATSPHVPKFSPEKFPLAGLEENYCRNPDNDENGPWCYTTDPDKRYDYCDIPECEDKCMHCSGENYEGKIAKTMSGRDCQAWDSQSPHAHGYIPSKFPNKNLKMNYCRNPDGEPRPWCFTTDPQKRWEFCDIPRCTTPPPSSGPKYQCLKGTGKNYGGTVAVTESGHTCQRWSEQTPHKHNRTPENFPCKNLEENYCRNPNGEKAPWCYTTNSEVRWEYCTIPSCESSPLSTERMDVPVPPEQTPVPQDCYHGNGQSYRGTSSTTITGRKCQSWSSMTPHRHLKTPENYPNAGLTMNYCRNPDADKSPWCYTTDPRVRWEFCNLKKCSETPEQVPAAPQAPGVENPPEADCMIGTGKSYRGKKATTVAGVPCQEWAAQEPHQHSIFTPETNPQSGLERNYCRNPDGDVNGPWCYTMNPRKPFDYCDVPQCESSFDCGKPKVEPKKCSGRIVGGCVSKPHSWPWQVSLRRSSRHFCGGTLISPKWVLTAAHCLDNILALSFYKVILGAHNEKVREQSVQEIPVSRLFREPSQADIALLKLSRPAIITKEVIPACLPPPNYMVAARTECYITGWGETQGTFGEGLLKEAHLPVIENKVCNRNEYLDGRVKPTELCAGHLIGGTDSCQGDSGGPLVCFEKDKYILQGVTSWGLGCARPNKPGVYVRVSPYVPWIEETMRRN

SEQ ID NO: 8 - protein, 333 aa, Canis familiaris
ASDCMFGNGKGYRGKKATTVMGIPCQEWAAQEPHRHSIFTPETNPQAGLEKNYCRNPDGDVNGPWCYTMNQRKLFDYCDVPQCVSTSFDCGKPQVEPKKCPGRVVGGCVANPHSWPWQISLRTRYGKHFCGGTLISPEWVLTAAHCLERSSRPASYKVILGAHKEVNLESDVQEIEVYKLFLEPTRADIALLKLSSPAVITSKVIPACLPPPNYVVADRTLCYITGWGETQGTYGAGLLKEAQLPVIENKVCNRYEYLNGRVKSTELCAGNLAGGTDSCQGDSGGPLVCFEKDKYILQGVTSWGLGCARPNKPGVYVRVSRFVTWIEGIMRNN

SEQ ID NO: 9 - protein, 809 aa, Erinaceus europaeus
MQRKELVLLFLLFLQPGHGIPLDDYVTTQGASLCSSTKKQLSVGSTEECAVKCEKETSFICRSFQYHSKEQQCVIMAENSKSTPVLRMRDVILFEKKMYLSECKVGNGKYYRGTVSKTKTGLTCQKWSAETPHKPRFSPDENPSEGLDQNYCRNPDNDPKGPWCYTMDPEVRYEYCEIIQCEDECMHCSGQNYVGKISRTMSGLECQPWDSQIPHPHGFIPSKFPSKNLKMNYCRNPDGEPRPWCFTMDRNKRWEYCDIPRCTTPPPPSGPTYQCLMGNGEHYQGNVAVTVSGLTCQRWGEQSPHRHDRTPENYPCKNLDENYCRNPDGEPAPWCFTTNSSVRWEFCKIPDCVSSASETEHSDAPVIVPPEQTPVVQECYQGNGQTYRGTSSTTITGKKCQPWTSMRPHRHSKTPENYPDADLTMNYCRNPDGDKGPWCYTTDPSVRWEFCNLKKCSGTEMSATNSSPVQVSSASESSEQDCIIDNGKGYRGTKATTGAGTPCQAWAAQEPHRHSIFTPETNPRADLQENYCRNPDGDANGPWCYTTNPRKLFDYCDIPHCVSPSSADCGKPKVEPKKCPGRVGGCVAHPHSWPWQVSLRRFGQHFCGGTLISPEWVVTAAHCLEKFSNPAIYKVVLGAHQETRLERDVQIKGVTKMFLEPYRADIALLKLSSPAIITDKDHPACLPNSNYMVADRSLCYITGWGETKGTYGAGLLKEAQLPVIEKVCNRQSFLNGRVRSTELCAGHLAGGVDSCQGDSGGPLVCFEKDRYILQGVTSWGLGCARLTRPGVYVRVSRYVSWLQDVMRNN

SEQ ID NO: 10 - protein, 338 aa, Equus caballus
VQEPSEPDCMLGIGKGYQGKKATTVTGTRCQAWAAQEPHRHSIFTPEANPWANLEKNYCRNPDGDVNGPWCYTMNPQKLFDYCDVPQCESSPFDCGKPKVEPKKCSGRIVGGCVAIAHSWPWQISLRTRFGRHFCGGTLISPEWVLTAAHCLERSSRPSTYKVVLGTHHELRLAAGAQQIDVSKLFLEPSRADIALLKLSSPAIITQNVIPACLPPADYVVANWAECFVTGWGETQDSSNAGVLKEAQLPVIENKVCNRYEYLNGRVKSTELCAGHLVGGVDSCQGDSGGPLVCFEKDKYILQGVTSWGLGCARPNKPGVYVRVSSFINWIERIMQSN

SEQ ID NO: 11 - protein, 810 aa, Macaca mulatta
MEHKEVVLLLLLFLKSGQGEPLDDYVNTKGASLFSITKKQLGAGSIEECAAKCEEEEEFTCRSFQYHSKEQQCVIMAENRKSSIVFRMRDVVLFEKKVYLSECKTGNGKNYRGTMSKTRTGITCQKWSSTSPHRPTFSPATHPSEGLEENYCRNPDNDGQGPWCYTTDPEERFDYCDIPECEDECMHCSGENYDGKISKTMSGLECQAWDSQSPHAHGYIPSKFPNKNLKKNYCRNPDGEPRPWCFTTDPNKRWELCDIPRCTTPPPSSGPTYQCLKGTGENYRGDVAVTVSGHTCHGWSAQTPHTHNRTPENFPCKNLDENYCRNPDGEKAPWCYTTNSQVRWEYCKIPSCESSPVSTEPLDPTAPPELTPVVQECYHGDGQSYRGTSSTTTTGKKCQSWSSMTPHWHEKTPENFPNAGLTMNYCRNPDADKGPWCFTTDPSVRWEYCNLKKCSGTEGSVAAPPPVAQLPDAETPSEEDCMFGNGKGYRGKKATTVTGTPCQEWAAQEPHSHRIFTPETNPRAGLEKNYCRNPDGDVGGPWCYTTNPRKLFDYCDVPQCAASSFDCGKPQVEPKKCPGRVVGGCVAYPHSWPWQISLRTRLGMHFCGGTLISPEWVLTAAHCLEKSSRPSFYKVILGAHREVHLEPHVQEIEVSKMFSEPARADIALLKLSSPAIITDKVIPACLPSPNYVVADRTECFITGWGETQGTYGAGLLKEARLPVIENKVCNRYEFLNGTVKTTELCAGHLAGGTDSCQGDSGGPLVCFEKDKYILQGVTSWGLGCARPNKPGVYVRVSRFVTWIEGVMRNN

SEQ ID NO: 12 - protein, 812 aa, Mus musculus
MDHKEVILLFLLLLKPGQGDSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRCSETGGSVVELPTVSQEPSGPSDSETDCMYGNGKDYRGKTAVTAAGTPCQGWAAQEPHRHSIFTPQTNPRADLEKNYCRNPDGDVNGPWCYTTNPRKLYDYCDIPLCASASSFECGKPQVEPKKCPGRVVGGCVANPHSWPWQISLRTRFTGQHFCGGTLIAPEWVLTAAHCLEKSSRPEFYKVILGAHEEYIRGLDVQEISVAKLILEPNNRDIALLKLSRPATITDKVIPACLPSPNYMVADRTICYITGWGETQGTFGAGRLKEAQLPVIENKVCNRVEYLNNRVKSTELCAGQLAGGVDSCQGDSGGPLVCFEKDKYILQGVTSWGLGCARPNKPGVYVRVSRFVDWIEREMRNN

SEQ ID NO: 13 - protein, 790 aa, Sus scrofa
DSLDDYVNTQGAFLFSLSRKQVAARSVEECAAKCEAETNFICRAFQYHSKDQQCVVMAENSKTSPIARMRDVVLFEKRIYLSECKTGNGKNYRGTTSKTKSGVICQKWSVSSPHIPKYSPEKFPLAGLEENYCRNPDNDEKGPWCYTTDPETRFDYCDIPECEDECMHCSGEHYEGKISKTMSGIECQSWGSQSPHAHGYLPSKFPNKNLKMNYCRNPDGEPRPWCFTTDPNKRWEFCDIPRCTTPPPTSGPTYQCLKGRGENYRGTVSVTASGHTCQRWSAQSPHKHNRTPENFPCKNLEENYCRNPDGETAPWCYTTDSEVRWDYCKIPSCGSSTTSTEHLDAPVPPEQTPVAQDCYRGNGESYRGTSSTTITGRKCQSWVSMTPHRHEKTPGNFPNAGLTMNYCRNPDADKSPWCYTTDPRVRWEYCNLKKCSETEQQVTNFPAIAQVPSVEDLSEDCMFGNGKRYRGKRATTVAGVPCQEWAAQEPHRHSIFTPETNPRAGLEKNYCRNPDGDDNGPWCYTTNPQKLFDYCDVPQCVTSSFDCGKPKVEPKKCPARVVGGCVSIPHSWPWQISLRYRYRGHFCGGTLISPEWVLTAKHCLEKSSSPSSYKVILGAHEEYHLGEGVQEIDVSKLFKEPSEADIALLKLSSPAVITDKVIPACLPTPNYVVADRTACYITGWGETKGTYGAGLLKEARLPVIENKVCNRYEYLGGKVSPNELCAGHLAGGIDSCQGDSGGPLVCFEKDKYILQGVTSWGLGCALPNKPGVYVRVSRFVTWIEEIMRRN

SEQ ID NO: 14 - DNA, 10 bp, Artificial Sequence
  Description of Artificial Sequencesynthetic, hypothetical
hawaaugucu

SEQ ID NO: 15 - protein, 4 aa, Artificial Sequence
  Description of Artificial Sequencesynthetic, substrate
AAPV

SEQ ID NO: 16 - protein, 4 aa, Artificial Sequence
  Description of Artificial Sequencesynthetic, substrate
AAPA

SEQ ID NO: 17 - protein, 18 aa, Homo sapiens
RNPDGDVGGPWCYTTNPR